(19) United States
(12) Patent Application Publication
(10) Pub. No.: US 2025/0082714 A1
(43) Pub. Date: Mar. 13, 2025

(54) METHODS AND COMPOSITIONS FOR THE TREATMENT OR PREVENTION OF MUSCULAR DYSTROPHY

(71) Applicant: Stealth BioTherapeutics Inc.

(72) Inventors: David Brown, Needham, MA (US); Dennis Keefe, Needham, MA (US)

(73) Assignee: Stealth BioTherapeutics Inc., (US)

(21) Appl. No.: 18/736,778

(22) Filed: Jun. 7, 2024

Related U.S. Application Data
(63) Continuation of application No. PCT/US2022/052239, filed on Dec. 8, 2022.
(60) Provisional application No. 63/287,812, filed on Dec. 9, 2021.

(51) Int. Cl. A61K 38/06; A61K 31/675; A61K 47/64; A61P 21/00

(52) U.S. Cl. CPC A61K 38/06; A61K 31/675; A61K 47/646; A61P 21/00

(57) ABSTRACT

The disclosure provides methods for treating muscular dystrophy (MD), such as Duchenne muscular dystrophy (DMD) or Becker muscular dystrophy (BMD) in a mammalian subject. The methods comprise administering to the subject an effective amount of a peptide such as H-D-Arg-2,6-Dmt-Lys-PHe-NH₂ (SEQ ID NO: 2) (a.k.a. MTP-131 or elamipretide), or a pharmaceutically acceptable salt, hydrate, solvate and/or tautomer thereof, in combination with at least one phosphorodiamidate morpholino oligomer (PMO) or peptide-conjugated PMO (PPMO). The methods are particularly useful for increasing dystrophin expression levels in subjects diagnosed with, and/or being treated for, MD in an amount greater than what would be possible by treatment with the PMO or PPMO alone.

### CROSS-REFERENCE TO RELATED APPLICATIONS

This application is a continuation of International Application No. PCT/US2022/052239, filed Dec. 8, 2022, and claims the benefit of and priority to U.S. Provisional Patent Application No. 63/287,812, filed Dec. 9, 2021, the entire contents of which are incorporated herein by reference.

### SEQUENCE LISTING

The instant application contains a Sequence Listing which has been submitted electronically in XML format and is hereby incorporated by reference in its entirety. Said XML copy, created on Nov. 25, 2024, is named 091151-1584_SL.xml and is 12,461 bytes in size.

### TECHNICAL FIELD

The present disclosure relates generally to compositions (e.g., medicaments or formulations), methods, and uses for treating or preventing Duchenne muscular dystrophy (DMD) or Becker's muscular dystrophy (BMD) that result from a subject's impaired ability to produce the protein, dystrophin. The present technology relates to administering, for example, an effective amount of a peptide and/or mixture of peptides such as H-D-Arg-2′6′-Dmt-Lys-Phe-NH₂ (SEQ ID NO: 2) (more commonly known as elamipretide, SS-31, MTP-131, or bendavia); or a pharmaceutically acceptable salt, hydrate, solvate and/or tautomer thereof, and/or its carboxylate form, H-D-Arg-2′6′-Dmt-Lys-Phe-OH (SEQ ID NO: 3) (or a pharmaceutically acceptable salt, hydrate, solvate and/or tautomer thereof) in combination with an effective amount of a phosphorodiamidate morpholino oligomer (PMO) or a peptide-conjugated PMO (PPMO), to a subject suffering from DMD or BMD.

### BACKGROUND

The following description is provided to assist the understanding of the reader. None of the information provided or references cited is admitted as prior art to the compositions and methods disclosed herein.

Muscular dystrophy (MD) is a group of inherited non-inflammatory but progressive muscle disorders. Duchenne muscular dystrophy (DMD) is the most common muscular dystrophy affecting 1 in about 3500 males born worldwide. Becker's muscular dystrophy (BMD) is milder than DMD and primarily causes heart problems. BMD affects only males (1 in about 30,000), usually first appears between the ages of 2 and 16 years but can appear as late as age 25. Both DMD and BMD result from abnormal or deficient production of the protein, dystrophin.

DMD begins with progressive muscle weakness that evolves to loss of ambulation and further progresses to early morbidity and mortality. DMD is caused by mutations in the dystrophin gene at locus Xp21, located on the short arm of the X chromosome. Dystrophin encodes a 427-kD protein that plays an integral role in the structural stability of the myofiber. The loss of dystrophin disrupts the muscle membrane and fibers. Without dystrophin, muscle fibers are susceptible to mechanical injury and necrotic/apoptotic cell death.

DMD is a progressive disease which eventually affects all voluntary muscles as well as cardiac and breathing muscles in later stages. The disease is most prevalent in males. While female carriers of the DMD mutation are largely asymptomatic, some (20-30%) present with mild to moderate muscle weakness and are at increased risk for developing DCM. Boys generally present with symptoms between the ages of three to five years. These symptoms generally worsen over time leading to loss of ambulation and the need for a wheelchair by early adolescence. Further progression of DMD leads to respiratory distress and cardiomyopathies, which is present in almost all males by the age of 18. The average life expectancy for individuals afflicted with DMD is around age 25.

Signs and symptoms of DMD include progressive proximal weakness with onset in the legs and pelvis, hyperlordosis with wide-based gait, hypertrophy of weak muscles, pseudohypertrophy (enlargement of calf and deltoid muscles with fat and fibrotic tissue), reduced muscle contractility on electrical stimulation in advanced stages of the disease, delayed motor milestones, progressive inability to ambulate, heel cord contractures, paralysis, fatigue, skeletal deformities including scoliosis, muscle fiber deformities, cardiomyopathy, congestive heart failure or arrhythmia, muscular atrophy, respiratory disorders, bladder or bowel dysfunction, sensory disturbance, or febrile illness. Weakness of skeletal muscle can contribute to cardiopulmonary complications. Scoliotic deformity from paraspinal muscle asymmetric atrophy can impair pulmonary and gastrointestinal function, predisposing individuals to pneumonia, respiratory failure, and poor nutrition. Smooth muscle dysfunction as a result of abnormal or absent dystrophin, along with inactivity, leads to gastrointestinal dysmotility, causing constipation and diarrhea.

DMD can be diagnosed in several ways. A clinical diagnosis may be made when a male child has progressive symmetrical muscle weakness. Muscle biopsy is an important tool for quantifying the amount of muscle dystrophin as well as for detecting asymptomatic female carriers of DMD. Immunostaining of the muscle using antibodies directed against the rod domain, carboxy-terminals, and amino-terminals of dystrophin protein shows absence of the usual sarcolemma staining in boys with DMD. A combination of clinical findings, family history, blood concentration of creatine phosphokinase and muscle biopsy with dystrophin studies confirms the diagnosis (Creatine phosphokinase is normally present in high concentrations in the muscle cells). However, DMD patients exhibit creatine phosphokinase levels that are 50-100 times the reference range (as high as 20,000 mU/mL) during the early stages of the disease). Electromyography, electrocardiogram and echocardiogram, and lung monitoring tests may be used for confirmatory diagnosis of DMD. The progression of DMD occurs in 5 stages: presymptomatic, early ambulatory, late ambulatory, early nonambulatory, and late nonambulatory.

As with other aspects of DMD, the cardiomyopathies are progressive but generally end with heart failure. Ultrasonography can detect structural changes in the myocardium well before the onset of systolic dysfunction and overt cardiomyopathy. Despite the high incidence of heart failure, the majority of children with DMD are relatively asymptomatic until late in the disease course, probably because of their inability to exercise. Heart failure and arrhythmias may develop in the late stages of the disease, especially during intercurrent infections or surgery. The late-stage cardiomyopathy is characterized by extensive fibrosis of the posterobasal left ventricular wall followed by spread of the fibrosis to the lateral free wall of the left ventricle. The continued progression of the cardiomyopathy often leads to output failure and pulmonary congestion. Alternatively, cardiac fibrosis can include cardiomyopathy and conduction abnormalities, which can induce fatal arrhythmias. Heart failure is the most common cause of death of persons afflicted with DMD.

Myocardial energy homeostasis is disrupted in DMD, with dysfunctional mitochondria being a central factor. Impaired mitochondrial function in the dystrophic heart is observed early in both animal DMD models and human studies, often before observable declines in cardiac function. The lack of dystrophin leads to cellular membrane fragility and heightened susceptibility to membrane rupture.

The loss of cell membrane integrity induces “leaky” fluxes of ions, enzymes, and metabolites. The influx of calcium is particularly problematic in the heart, which requires tightly regulated calcium cycling for pump function. Calcium content is normally three to four orders of magnitude lower in the cytosol compared to the outside of the cell. Heightened calcium within DMD myocytes causes sarcomeric disruption and calcium overload in mitochondria.

Mitochondrial calcium overload leads to several inter-related problems in the DMD heart. Calcium overload opens the mitochondrial permeability transition pore, a non-specific mitochondrial channel that can initiate apoptotic cell death. Opening of this pore can be catastrophic for mitochondria, as it collapses electrochemical and metabolite gradients that are crucial for ATP generation. DMD mitochondria have heightened production of reactive oxygen species, which can exacerbate cellular damage. Ruptured mitochondrial fragments can leak out of cells and contribute to inflammatory signaling cascades. Finally, mitochondrial structure, which is directly related to bioenergetic function, is compromised in DMD.

Mitochondrial dysfunction in DMD is a key contributor to cellular death. As the regenerative capacity of the heart is very low, the loss of myocytes places an increased burden on the surviving cells. Mitochondria within viable cells are under heightened pressure to meet the constant ATP demands of the heart. Futile pathological cycles continue to overwhelm cellular defense mechanisms as the disease progresses. Ensuing cardiac remodeling leads to higher propensity for electromechanical dysfunction and ultimately compromised cardiac function.

Historically, DMD and BMD patients have been treated for their symptoms. The standard of care has been treatment with corticosteroids to increase muscle function, ACE inhibitors, ARBs and beta blockers to address the progressive cardiomyopathy and assistive devices to address ambulatory needs. More recently, phosphorodiamidate morpholino oligomers (PMOs), which are drugs based on an exon skipping mechanism of action that are directed to upregulation in the expression of the protein, dystrophin, in DMD patients (i.e., Exondys 51® (Eteplirsen), Vyondys 53™ (Golodirsen), Viltepso® (Viltolarsen), or Amondys 45™ (Casimersen)), have been approved by the United States Food and Drug Administration (FDA). While these drugs appear to increase the expression of dystrophin in skeletal muscle, a key limitation to the efficacy of PMOs is variability in their potency, tissue uptake and retention, and tissue specificity (notably low in cardiac tissue).

In summary, although there are current treatments for symptoms and recent advances in medicine have begun to address the molecular underpinnings of the disease, MD (including, without limitation, DMD and BMD) remains an incurable illness for which additional treatments and therapies are desperately needed.

### SUMMARY

In one aspect, the disclosure of the present technology provides a method for treating muscular dystrophy, comprising administering to the subject a therapeutically effective amount of a phosphorodiamidate morpholino oligomer (PMO) or a peptide-conjugated PMO (PPMO) in combination with a peptide of formula A:

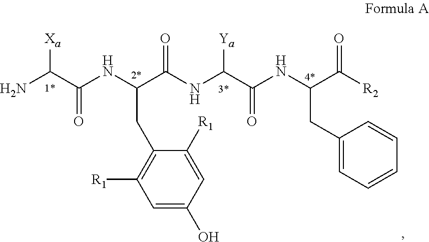

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

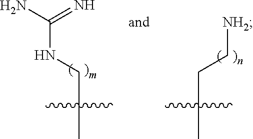

each m is 2, 3 or 4; each n is independently 1, 2, or 3; and the absolute stereochemistry at each of stereocenters 1*, 2*, 3*, and 4* is independently D or L.

In some embodiments, the peptide of generic Formula A is a peptide of formula A-1:

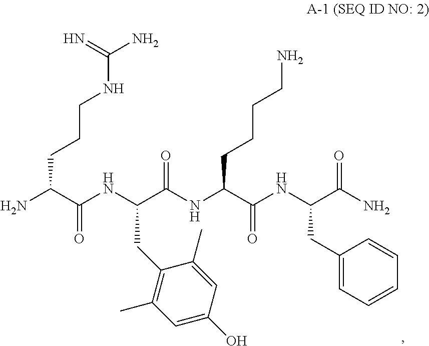 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

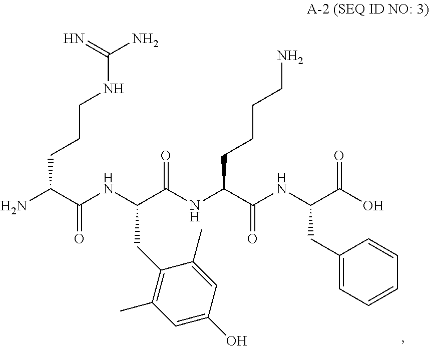 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-3, A-4, A-5, A-6, A-7 or A-8:

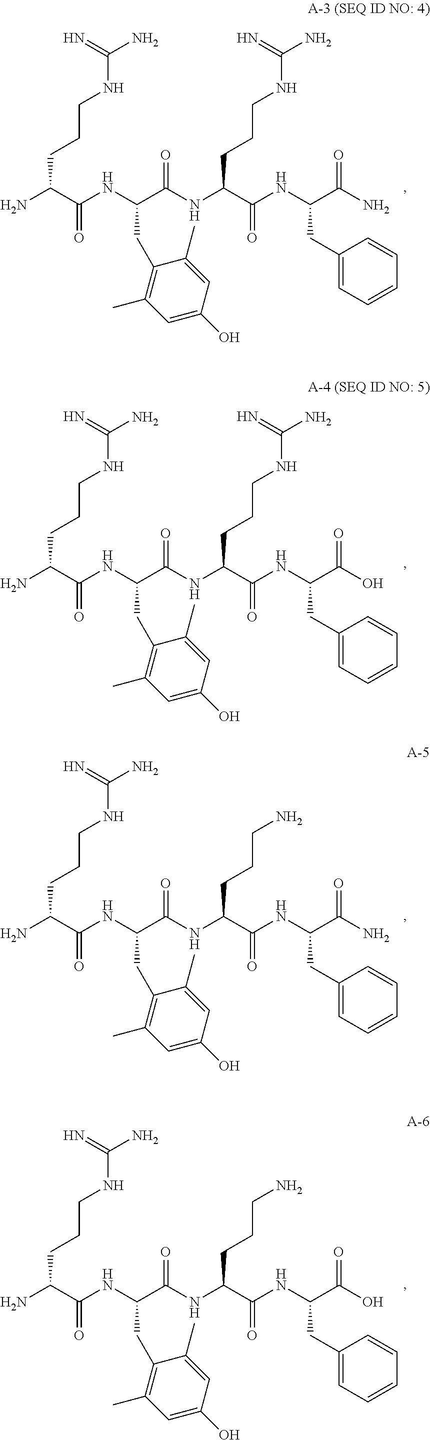 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

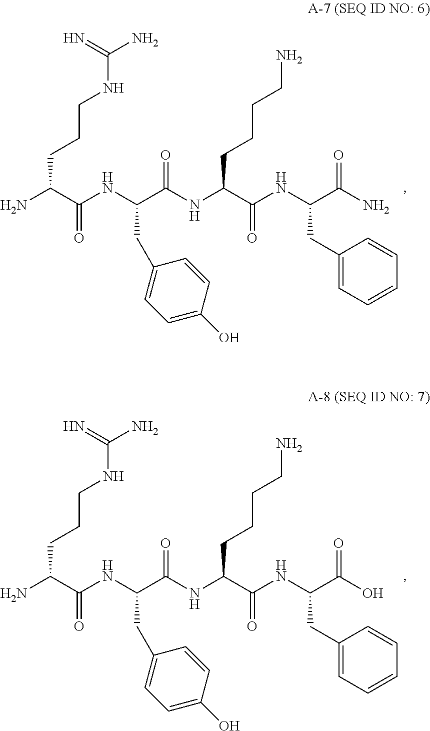 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O ; N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate, and/or tautomer thereof.

In some embodiments, treatment reduces, ameliorates, and/or delays the onset of muscular dystrophy. In some embodiments, the administration increases the level of dystrophin expression in the subject compared to a control subject. In some embodiments, the administration increases dystrophin expression in skeletal, cardiac, and/or smooth muscle in the subject as compared to an untreated subject (or untreated control group of subjects) or as compared to a subject (or control group of subjects) administered the PMOs and/or PPMOs alone or the peptide (or mixture of peptides) alone. In some embodiments, the muscular dystrophy is Duchenne muscular dystrophy. In some embodiments, the muscular dystrophy is Becker's muscular dystrophy. In some embodiments, the peptide is administered daily for: (i) 24 weeks or more; (ii) 48 weeks or more; (iii) 72 weeks or more; or (iv) 96 weeks or more. In some embodiments, the PMO or PPMO is administered once weekly for: (i) 24 weeks or more; (ii) 48 weeks or more; (iii) 72 weeks or more; or (iv) 96 weeks or more. In some embodiments, the peptide and PMO or PPMO are administered orally, topically, systemically, intraperitoneally, subcutaneously, intravenously, intradermally, transdermally, ophthalmically, intrathecally, intracerebroventricularly, iontophoretically, transmucosally, intravitreally, intranasally, or intramuscularly. In some embodiments, the PMO or PPMO is administered intravenously. In some embodiments, the peptide is administered subcutaneously. In some embodiments, the subject is human.

In some embodiments, the method further comprises separately, sequentially, or simultaneously administering an additional therapeutic agent to the subject.

In some embodiments, the PMO is selected from the group consisting of Eteplirsen (Exondys 51®), Golodirsen (Vyondys 53™), Viltolarsen (Viltepso®), and Casimersen (Amondys 45™).

In some embodiments, the peptide and the PMO or PPMO are administered intravenously. In some embodiments, the peptide and the PMO or PPMO are administered simultaneously.

In some embodiments, the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

In some embodiments, the peptide of Formula A is administered in a depot formulation. In some embodiments, the depot formulation comprises the peptide of Formula A encapsulated or otherwise disposed in silica microparticles. In some embodiments, the depot formulation is a sustained release depot formulation. In some embodiments, the peptide of Formula A is released in an effective amount over days, weeks or months.

In one aspect, the disclosure of the present technology provides a composition comprising:
a) a peptide of formula A:

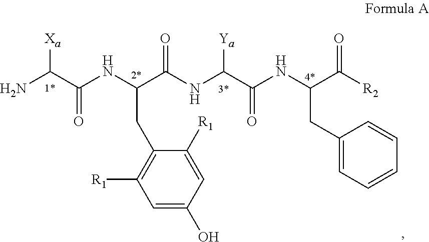

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

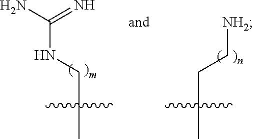

each m is 2, 3 or 4; each n is independently 1, 2, or 3; and the absolute stereochemistry at each of stereocenters 1*, 2*, 3*, and 4* is independently D or L; and
b) a phosphorodiamidate morpholino oligomer (PMO) or a peptide-conjugated PMO (PPMO).

In some embodiments, the peptide is A-1 (SEQ ID NO: 2) or A-2 (SEQ ID NO: 3):

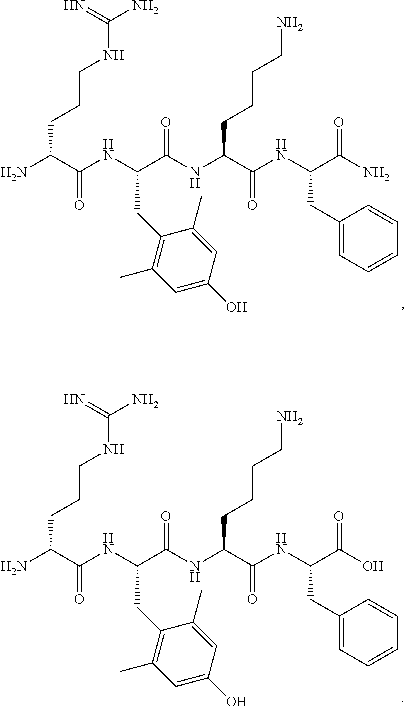 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

In some embodiments, the PMO is selected from the group consisting of Eteplirsen (Exondys 51®), Golodirsen (Vyondys 53™), Viltolarsen (Viltepso®), and Casimersen (Amondys 45™).

In some embodiments, the composition is a medicament.

In some embodiments, the composition is formulated for intravenous administration.

In some embodiments, the composition is a depot formulation. In some embodiments, the depot formulation comprises the peptide of Formula A and the PMO or PPMO encapsulated or otherwise disposed in silica microparticles. In some embodiments, the depot formulation is a sustained release depot formulation.

In one aspect, the disclosure of the present technology provides a method for augmenting the production of dystrophin in a mammalian subject in need thereof, comprising administering to the subject a therapeutically effective amount of a phosphorodiamidate morpholino oligomer (PMO) or a peptide-conjugated PMO (PPMO) in combination with a peptide of formula A:

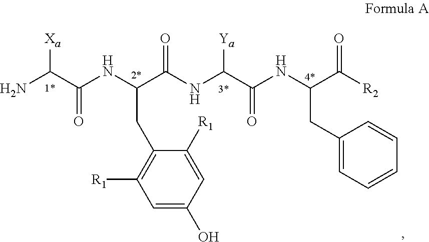

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

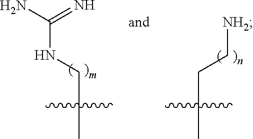

each m is 2, 3 or 4; each n is independently 1, 2, or 3; and the absolute stereochemistry at each of stereocenters 1*, 2*, 3*, and 4* is independently D or L, wherein the administration increases dystrophin expression in skeletal, cardiac, and or smooth muscle in the subject as compared to an untreated subject (or untreated control group of subjects) or as compared to a subject (or control group of subjects) administered the PMO and/or PPMO alone or the peptide alone.

In some embodiments, the peptide of generic Formula A is a peptide of formula A-1:

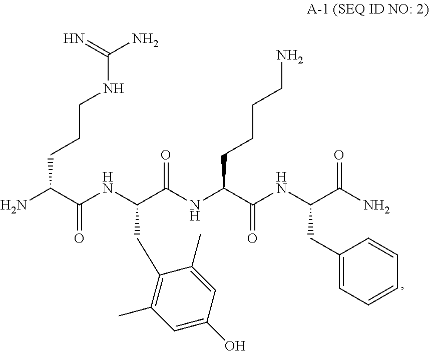 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

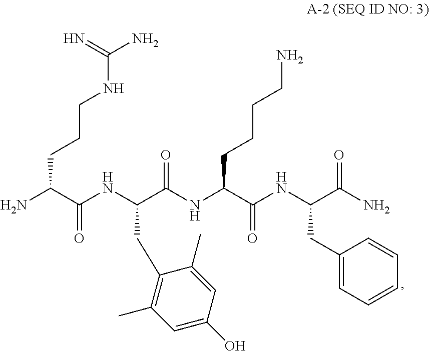 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-3, A-4, A-5, A-6, A-7 or A-8:

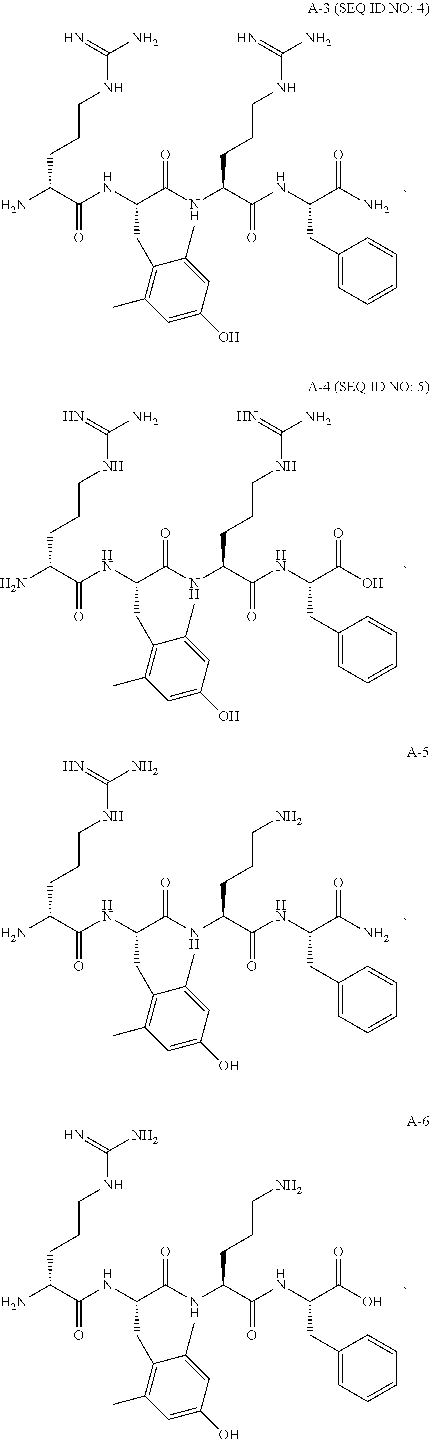 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

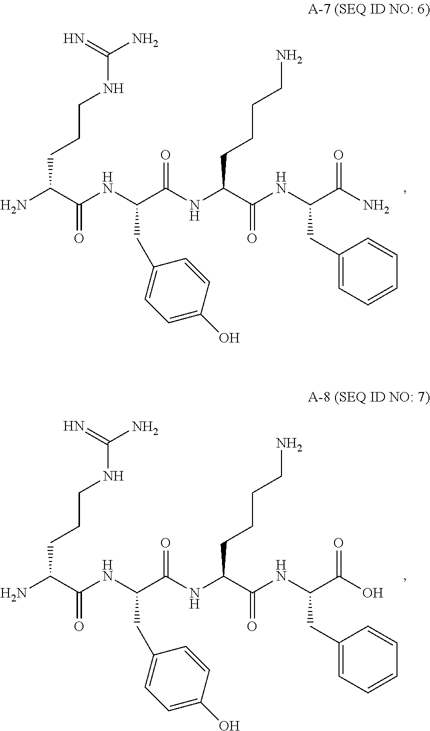 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O ; N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate, and/or tautomer thereof.

In some embodiments, the subject in need thereof has been diagnosed with, is suspected of having, or is at risk for developing muscular dystrophy.

In some embodiments, treatment reduces, ameliorates, and/or delays the onset of muscular dystrophy.

In some embodiments, the muscular dystrophy is Duchenne muscular dystrophy (DMD.

In some embodiments, the muscular dystrophy is Becker's muscular dystrophy (BMD).

In some embodiments, the peptide is administered daily for: (i) 24 weeks or more; (ii) 48 weeks or more; (iii) 72 weeks or more; or (iv) 96 weeks or more.

In some embodiments, the PMO or PPMO is administered once weekly for: (i) 24 weeks or more; (ii) 48 weeks or more; (iii) 72 weeks or more; or (iv) 96 weeks or more.

In some embodiments, the peptide and PMO or PPMO are administered orally, topically, systemically, intraperitoneally, subcutaneously, intravenously, intradermally, transdermally, ophthalmically, intrathecally, intracerebroventricularly, iontophoretically, transmucosally, intravitreally, intranasally, or intramuscularly.

In some embodiments, the PMO or PPMO is administered intravenously.

In some embodiments, the peptide is administered subcutaneously.

In some embodiments, the subject is human.

In some embodiments, the method further comprises separately, sequentially, or simultaneously administering an additional therapeutic agent to the subject.

In some embodiments, the PMO is selected from the group consisting of Eteplirsen (Exondys 51®), Golodirsen (Vyondys 53™), Viltolarsen (Viltepso®), and Casimersen (Amondys 45™).

In some embodiments, the peptide and the PMO or PPMO are administered intravenously.

In some embodiments, the peptide and the PMO or PPMO are administered simultaneously.

In some embodiments, the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

In some embodiments, the peptide of Formula A is administered in a depot formulation. In some embodiments, the depot formulation comprises the peptide of Formula A encapsulated or otherwise disposed in silica microparticles. In some embodiments, the depot formulation is a sustained release depot formulation. In some embodiments, the peptide of Formula A is released in an effective amount over days, weeks or months.

### BRIEF DESCRIPTION OF THE DRAWINGS

FIGS. 1A and 1B are charts showing the body weights (FIG. 1A) and body weight change (FIG. 1B) of mice from each of the treatment groups from 5 to 10 weeks of age. Data represented as Mean±SEM; n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)).

FIG. 1C is a chart showing the body weights of mice from each of the treatment groups at 10 weeks of age. Data represented as Mean with SEM; n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)).

FIGS. 1D and 1E are charts showing in vitro maximal force (FIG. 1D) and specific force (FIG. 1E) measurements on mouse right extensor digitorum longus (EDL) muscle at the end of the study (weeks 6-7; mice age 10-11 weeks). Data represented as Mean with SEM. Unpaired t-test (Mdx Vehicle compared to BL10 Vehicle): ###: p<0.001; ##: p<0.01. One-Way ANOVA (each treatment group compared to mdx Vehicle): ns: non-significant p>0.05. For maximal and specific force 1-2 values per group removed due to technical error. For Specific Force, 1 value removed due to outlier (Mdx vehicle: 1×PBS+0.9% saline).

FIGS. 1F and 1G are charts showing absolute (FIG. 1F) and normalized (FIG. 1G) mouse heart tissue weights following study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): #: p<0.05 Abs. Heart, ns: non-significant p>0.05 Norm. Heart. One-Way ANOVA (each treatment group compared to mdx Vehicle): ns: non-significant p>0.05 for Abs. Heart and Norm. Heart. Data represented as Mean with SEM.

FIGS. 1H and 1I are charts showing average and normalized mouse tibialis anterior (TA) tissue weights measured at study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): ###: p<0.001. One-Way ANOVA (each treatment group compared to mdx Vehicle): ns: non-significant p>0.05. Data represented as Mean with SEM.

FIGS. 1J and 1K are charts showing average and normalized mouse extensor digitorum longus (EDL) tissue weights measured at study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): ###: p<0.001. One-Way ANOVA (each treatment group compared to mdx Vehicle): **: p<0.01; ns: non-significant p>0.05. Data represented as Mean with SEM.

FIGS. 1L and 1M are charts showing average and normalized mouse quadriceps (quad) tissue weights measured at study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): ###: p<0.001. One-Way ANOVA (each treatment group compared to mdx Vehicle): **: p<0.01; ***: p<0.001; ns: non-significant p>0.05. Data represented as Mean with SEM.

FIGS. 1N and 1O are charts showing average and normalized mouse gastrocnemius (gastroc) tissue weights measured at study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): ###: p<0.001. One-Way ANOVA (each treatment group compared to mdx Vehicle): *: p<0.05; ***: p<0.001; ns: non-significant p>0.05. Data represented as Mean with SEM.

FIGS. 1P and 1Q are charts showing average and normalized mouse soleus tissue weights at study completion. n=15 for groups 1 (BL10 vehicle: 1×PBS+0.9% Saline) and 5 (PMO (125 mg/kg)+MTP-131 (5 mg/kg)); n=14 for groups 2 (MDX vehicle: 1×PBS+0.9% Saline), 3 (PMO(125 mg/kg)+0.9% Saline), and 4 (1×PBS+MTP-131 (5 mg/kg)). T-test (mdx Vehicle compared to BL10 Vehicle): ###: p<0.001. One-Way ANOVA (each treatment group compared to mdx Vehicle): *: p<0.05; ns: non-significant p>0.05. Data represented as Mean with SEM.

FIGS. 1R and 1S are charts showing the percent inflammation in mouse tibialis anterior (TA) tissues. FIG. 1R: n=8 per group. % decrease in inflammation shown for treatment group compared to Mdx vehicle. FIG. 1S: % inflammation per treatment group with 1 outlier removed from group 3 (PMO (125 mg/kg)+0.9% Saline). n=8 for group 1 (BL10 vehicle: 1×PBS+0.9% Saline), 2 (MDX vehicle: 1×PBS+0.9% Saline), 4 (1×PBS+MTP-131 (5 mg/kg) and 5 (PMO (125 mg/kg)+MTP-131 (5 mg/kg)). n=7 for group 3 (PMO (125 mg/kg)+0.9% Saline). % decrease in inflammation shown for each treatment group compared to Mdx vehicle. T-test (mdx Vehicle compared to BL10 Vehicle): ###: p<0.001. One-Way ANOVA (each treatment group compared to Mdx vehicle): *: p<0.05. ns: non-significant p>0.05. Data represented as Median.

FIGS. 1T-1X are images of Hematoxylin & Eosin (H&E) staining of mouse tibialis anterior (TA) muscle sections taken at study completion and analyzed for percent inflammation. FIG. 1T: BL10 Vehicle (1×PBS+0.9% Saline): mouse ID 2792; FIG. 1U: MDX Vehicle (1×PBS+0.9% Saline): mouse ID 2791; FIG. 1V: PMO (125 mg/kg)+0.9% Saline: mouse ID 2726; FIG. 1W: MTP-131 (5 mg/kg)+1×PBS: mouse ID 2744; FIG. 1X: PMO (125 mg/kg)+MTP-131 (5 mg/kg): mouse ID 2734.

FIGS. 2A-2L are Western blots (WB) showing the expression of dystrophin in mouse tibialis anterior (TA) muscle sections at study completion (FIG. 2A (WB TA Run 1, Gel & Blot 5), 2C (WB TA Run 1, Gel & Blot 6), 2E (WB TA Run 2, Gel & Blot 7), 2G (WB TA Run 2, Gel & Blot 8), 2I (WB TA Run 3, Gel & Blot 9), 2K (WB TA Run 3, Gel & Blot 10)), along with their respective standard curves (FIGS. 2B, 2D, 2F, 2H, 2J, 2L), for each of the mice (designated by mouse ID number) included in each of the study groups (“MDX Vehicle” (1×PBS+0.9% Saline); “PMO+Saline” (PMO (125 mg/kg)+0.9% Saline)); “MTP-131+PBS” (MTP-131 (5 mg/kg)+1×PBS); and “PMO+MTP-131” (PMO (125 mg/kg)+MTP-131 (5 mg/kg)). *in FIG. 2C=Samples 2740 and 2758 were repeated due to bubble on this blot. 2758 was repeated in Run 2 blot 8. 2740 was repeated in Run 2 blot 7 (undiluted) and was then run again in Run 3 blot 10 (diluted 1:2). *in FIG. 2E=2740 diluted 1:2 in Run 3 (Blot 10) as result was outside the standard curve range. *in FIG. 2G=2758 was repeated in this run. Originally ran in Run 1 Blot 6.

FIG. 2M is chart showing the % dystrophin in tibialis anterior (TA) tissue by treatment group. n=14 for groups 2 (MDX vehicle: 1×PBS+0.9% Saline), 3 (PMO (125 mg/kg)+0.9% Saline)), and 4 (1×PBS+MTP-131 (5 mg/kg)). n=15 for group 5 (PMO (125 mg/kg)+MTP-131 (5 mg/kg). Group 1 (BL10 vehicle: 1×PBS+0.9% Saline) not included for % dystrophin. T-test: MDX Vehicle compared to PMO+Saline, ***: p<0.001; Mdx vehicle compared to PMO+MTP-131, ***: p<0.001; PMO+Saline compared to PMO+MTP-131, *: p<0.05. Data represented as Mean with SEM.

FIGS. 2N-2R are immunofluorescence images of anti-dystrophin antibody staining of mouse tibialis anterior (TA) muscle sections taken at study completion. Representative images are provided for each treatment group. Encapsulation around the muscle fibers shows dystrophin, and punctate staining show nuclei. FIG. 2N: Healthy Mouse (BL10) Vehicle (1×PBS+0.9% Saline); FIG. 2O: MDX Vehicle (1×PBS+0.9% Saline); FIG. 2P: MDX PMO Alone (PMO (125 mg/kg)+0.9% Saline); FIG. 2Q: MDX Elamipretide Alone (MTP-131 (5 mg/kg)+1×PBS); FIG. 2R: MDX PMO+Elamipretide (PMO (125 mg/kg)+MTP-131 (5 mg/kg)).

FIG. 3 is an illustration of a peptide tetramer compound of general Formula A and two exemplary peptides A-1 (H-D-Arg-2,6-Dmt-Lys-Phe-NH₂ (SEQ ID NO: 2)) and A-2 (H-D-Arg-2,6-Dmt-Lys-Phe-OH (SEQ ID NO: 3)).

FIG. 4 is an illustration of various salt forms of the tetrapeptide of exemplary peptide A-2. FIG. 4 discloses Structures 21, 22, 20, 23, 24, and 25 as SEQ ID NO: 3.

FIG. 5 is an illustration of various salt forms of the tetrapeptide of exemplary peptide A-1 (elamipretide). Figure discloses Structures 31, 33, 34, and 35 as SEQ ID NO: 2.

### DEFINITIONS

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended claims, the singular forms “a”, “an” and “the” include plural references unless the content clearly dictates otherwise. For example, reference to “a cell” includes a combination of two or more cells, and the like.

As used herein, the “administering” or the “administration” of an agent (e.g., a peptide) or drug (e.g., PMO or PPMO) to a subject refers to any route of introducing or delivering to a subject a compound (e.g., peptide, mixture of peptides, or combination of peptide(s) and PMO or PPMO) to perform its intended function. Administration can be carried out by any suitable route, such as oral administration. Administration can be carried out subcutaneously. Administration can be carried out intravenously. Administration can be carried out intraocularly. Administration can be carried out retro-orbitally. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or the administration by a device (e.g., a pump).

As used herein, the term “amino acid” refers to naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein the term or phrase “carrier” and “pharmaceutically acceptable carrier” refer to a diluent, adjuvant, excipient, or vehicle with which a peptide/compound/composition is administered or formulated for administration. Non-limiting examples of such pharmaceutically acceptable carriers include liquids, such as water, saline, and oils; and solids, such as gum acacia, gelatin, starch paste, talc, keratin, colloidal silica, silica particles (nanoparticles or microparticles), urea, and the like. In addition, auxiliary, stabilizing, thickening, lubricating, flavoring, and coloring agents may be used. Other examples of suitable pharmaceutical carriers are described in Remington's Pharmaceutical Sciences by E.W. Martin, herein incorporated by reference in its entirety.

As used herein, the phrase “delaying the onset of” refers to, in a statistical sample, postponing, hindering, or causing one or more symptoms of a disorder, symptom, condition or indication to occur more slowly than normal in a treated sample relative to an untreated control sample.

As used herein, the term “effective amount” refers to a quantity of the peptide(s) and PMO(s) and/or PPMO(s) sufficient to achieve a desired therapeutic and/or prophylactic effect, e.g., an amount that treats, inhibits, reduces, ameliorates, or delays the onset of DMD or BMD when “co-administered,” where, for example, the peptide(s) and PMO(s) and/or PPMO(s) may be administered simultaneously, sequentially, or by separate administration. In the context of therapeutic or prophylactic applications, in some embodiments, the amount of a composition administered to the subject will depend on the type and severity of the disease and on the characteristics of the individual, such as general health, age, sex, body weight, and tolerance to drugs. The skilled artisan will be able to determine appropriate dosages depending on these and other factors. The peptide(s) and PMO(s) and/or PPMO(s) disclosed herein can be administered in an effective amount prior to the onset of one or more symptoms associated with DMD or BMD, or in response to a symptom that occurs in a subject suffering from DMD or BMD. The combination of peptide(s) and PMO(s) and/or PPMO(s) disclosed herein can also be administered in combination with one or more additional therapeutic compounds, and could be administered simultaneously, sequentially, or by separate administration. The one or more additional therapeutic compounds/compositions could be, for example, a corticosteroid, an ACE inhibitor, an ARB, and/or a beta-blocker. In some embodiments, the co-administration of a peptide (or mixture of peptides) and a PMO(s) and/or PPMO(s) may produce a synergistic therapeutic effect. In some embodiments, the co-administration of a peptide (or mixture of peptides) and a PMO(s) and/or PPMO(s), and one or more additional therapeutic compounds may produce a synergistic therapeutic effect.

In the methods described herein, therapeutic compounds (e.g., a peptide or mixture of peptides and PMO(s) and/or PPMO(s)), or pharmaceutically acceptable salts, stereoisomers, mixtures of stereoisomers, tautomers, hydrates, and/or solvates thereof, may be administered to a subject having one or more signs, symptoms, or risk factors of DMD, or BMD. For example, a “therapeutically effective amount” of therapeutic compounds (e.g., a peptide or mixture of peptides and PMO(s) and/or PPMO(s)) includes levels at which the presence, frequency, or severity of one or more signs, symptoms, or risk factors of DMD or BMD are inhibited, reduced or eliminated. In some embodiments, a therapeutically effective amount of therapeutic compounds (e.g., a peptide or mixture of peptides and PMO(s) and/or PPMO(s)) augments the production of dystrophin in the subject when compared to a control subject administered PMO(s) and/or PPMO(s) alone.

As used herein, the term “hydrate” refers to a compound (e.g., a peptide or mixture of peptides) which is associated with water. The number of the water molecules contained in a hydrate of a compound may be (or may not be) in a definite ratio to the number of the compound molecules in the hydrate.

As used herein, the term “inhibit,” “inhibits,” or “inhibiting” means to reduce by an objectively measurable amount or degree compared to control. In one embodiment, inhibit or inhibiting means reduce by at least a statistically significant amount compared to control. In some embodiments, inhibit or inhibiting means reducing by at least 1-5 percent compared to control. In various individual embodiments, inhibit or inhibiting means reducing by at least 2, 3, 4, 5, 6, 7, 8, 9, 10, 15, 20, 25, 30, 33, 40, 50, 60, 67, 70, 75, 80, 90, 95, or 99 percent compared to control.

As used herein, the term “separate” with respect to a therapeutic use refers to an administration of at least two active ingredients at the same time or at substantially the same time by different routes. The “active ingredients” can, for example, be a peptide or mixture of peptides as disclosed herein and at least one of a PMO (such as Eteplirsen (Exondys 51®), Golodirsen (Vyondys 53™), Viltolarsen (Viltepso®), or Casimersen (Amondys 45™)) or PPMO. In some embodiments, the active ingredients may further include one or more additional therapeutic compounds/compositions, for example, a corticosteroid, an ACE inhibitor, an ARB, and/or a beta-blocker.

As used herein, the term “sequential” with respect to a therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition. The “active ingredients” can, for example, be a peptide or mixture of peptides and at least one PMO (such as Eteplirsen (Exondys 51®), Golodirsen (Vyondys 53™), Viltolarsen (Viltepso®), or Casimersen (Amondys 45™)) and/or PPMO as disclosed herein. In some embodiments, the active ingredients may further include at least one of a corticosteroid, an ACE inhibitor, and or a beta-blocker.

As used herein, the term “simultaneous” with respect to a therapeutic use refers to the administration of at least two active ingredients (i.e., two pharmaceutically active ingredients) by the same or different route but at the same time or at substantially the same time. The “active ingredients” can, for example, be a peptide or mixture of peptides as disclosed herein and at least one PMO (such as Eteplirsen (Exondys 51®), Golodirsen (Vyondys 53™), Viltolarsen (Viltepso®), or Casimersen (Amondys 45™)) and/or PPMO. In some embodiments, the active ingredients may further include at least one of a corticosteroid, an ACE inhibitor, an ARB, and/or a beta-blocker.

As used herein, the term “subject” refers to a living animal. In various embodiments, a subject is a mammal. In some embodiments, a subject is a non-human mammal, including, without limitation, a mouse, rat, hamster, guinea pig, rabbit, sheep, goat, cat, dog, pig, minipig, horse, cow, or non-human primate. In some embodiments, the subject is a human.

As used herein, the term “treat”, “treats”, “treating” or “treatment” refers to therapeutic treatment, wherein the object is to reduce, alleviate or delay onset of the progression or advancement of, and/or reverse the progression of the targeted pathological condition or disorder.

As used herein, “peptide-conjugated PMOs (PPMOs)” are PMOs to which a cell penetrating peptide is linked in order to improve cellular uptake of the PMO. See: Tsoumpra et al. (2019) “Peptide-conjugated antisense based splice-correction for Duchenne muscular dystrophy and other neuromuscular diseases” EBioMedicine, 45, 630-645; doi.org/10.1016/j.ebiom.2019.06.036.

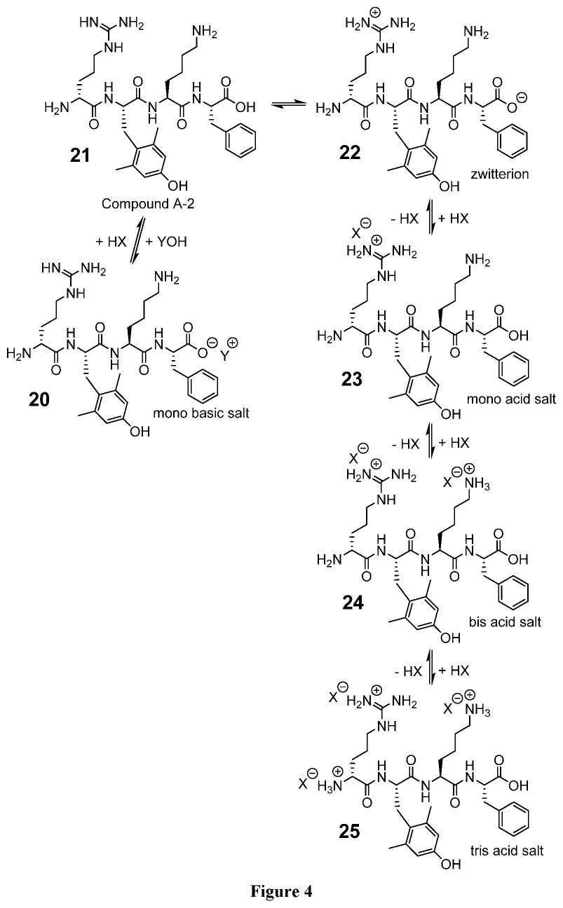

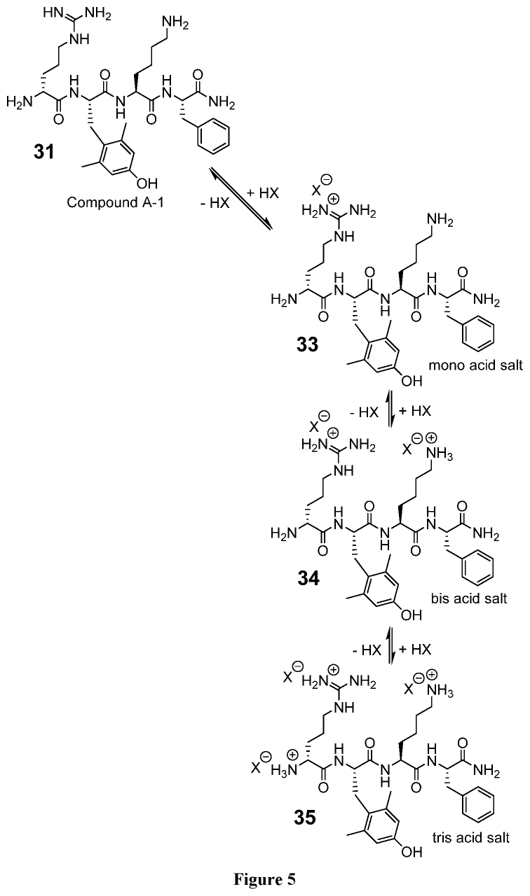

As used herein, the term “pharmaceutically acceptable salt” refers to a salt of a therapeutically active compound (e.g., a peptide or mixture of peptides and a PMO and/or PPMO) that can be prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts. These salts may be prepared by methods known to those skilled in the art. Other pharmaceutically acceptable carriers known to those of skill in the art are suitable for the present technology. In some embodiments, the compound is a zwitterion (an intramolecular salt). Exemplary salt forms of the peptide H-D-Arg-2′6′-Dmt-Lys-Phe-OH (SEQ ID NO: 3) (A-2) are illustrated in FIG. 4. Exemplary salt forms of the peptide H-D-Arg-2′6′-Dmt-Lys-Phe-NH₂ (SEQ ID NO: 2) (A-1) are illustrated in FIG. 5.

As used herein, “phosphorodiamidate morpholino oligomers (PMOs)” refer to synthetic oligomers comprising a natural nucleobase linked to methylenemorpholine rings linked through phosphorodiamidate groups instead of a phosphate backbone. See: Summerton J E (2017). “Invention and Early History of Morpholinos: From Pipe Dream to Practical Products”. Morpholino Oligomers. Methods in Molecular Biology. 1565. Humana Press (Springer). pp. 1-15. In some embodiments, the PMO comprises an appropriately designed exon-skipping oligomer that is relevant to the dystrophin lesion in a subject in need thereof, wherein the subject has been diagnosed with or is suspected of having a muscular dystrophy, such as DMD or BMD. By way of example, but not by limitation, the PMO comprises an antisense oligomer of about 20-50 nucleotides in length, or a pharmaceutically acceptable salt thereof, capable of binding a selected target in human dystrophin pre-mRNA to induce exon skipping in the human dystrophin gene, wherein the antisense oligomer comprises a sequence of bases that specifically hybridizes to a dystrophin exon target region. By way of example, but not by limitation, the PMO may be chemically linked to a cell penetrating peptide that improves cellular uptake of the PMO (e.g., a PPMO). Illustrative, non-limiting examples of PMOs include any one or more the PMOs selected from Eteplirsen (Exondys 51®), Golodirsen (Vyondys 53™), Viltolarsen (Viltepso®), and Casimersen (Amondys 45™). Two or more antisense oligomers may be used together to induce exon skipping of single or multiple exons.

As used herein the term “prevent”, “prevents”, “preventing” or “prevention” refers to, in a statistical sample, reducing the occurrence of a disorder, symptom, condition or indication in a treated sample relative to an untreated control sample.

As used herein the term “prophylactic” refers to an action intended to prevent a disorder, symptom, condition or indication from occurring.

As used herein, the term “solvate” refers to forms of a compound (e.g., a peptide or mixture of peptides) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, the terms “subject” and “patient” are used interchangeably.

As used herein the term “synergistic therapeutic effect” refers to a greater-than-additive therapeutic effect that is produced by a combination of at least two therapeutic agents, and which exceeds that which would otherwise result from the individual administration of the agents. For example, lower doses of one or more therapeutic agents may be used in treating DMD or BMD, resulting in increased therapeutic efficacy and decreased side-effects. In some embodiments, the co-administration of a peptide or mixture of peptides as disclosed herein (e.g., a peptide of Formula A, A-1, A-2, A-3, A-4, A-5, A-6, A-7, or A-8) augments PMO-mediated dystrophin expression in a synergistic manner.

As used herein, the term “tautomer” refers to compounds (e.g., a peptide or mixture of peptides) that are interchangeable forms of a particular compound structure, and that vary in the displacement of hydrogen atoms and electrons. Thus, two structures may be in equilibrium through the movement of T electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

### DETAILED DESCRIPTION

In one aspect, the present disclosure provides methods for treating DMD or BMD, or inhibiting the onset or progression of DMD or BMD in a mammalian subject suffering from DMD or BMD, comprising administering to the subject in need thereof a therapeutically effective amount of a peptide or mixture of peptides in combination with one or more PMOs and/or PPMOs as described in more detail below, or a pharmaceutically acceptable salt, hydrate, solvate and/or tautomer thereof. Generally, the mammalian subject will harbor a genetic permutation that affects the production and/or function of dystrophin protein. In some embodiments of the method, the genetic permutation is an insert, deletion, duplication, frameshift, or nonsense mutation related to the production of dystrophin protein. In some embodiments of the method, administering the peptide or mixture of peptides in combination with one or more PMOs and/or PPMOs to the subject results in an augmentation in the dystrophin expression in the subject as compared to a control subject administered the peptide or mixture of peptides alone or the PMO(s) and/or PPMO(s) alone. Said peptide or mixture of peptides in combination with one or more PMOs and/or PPMOs can be administered alone, in a composition or formulation (e.g., medicament), and/or in combination with one or more additional therapeutic agents/drugs (i.e. active ingredients). In some embodiments, the subject is human.

The peptide or peptides within a mixture of peptides can be of generic Formula A:

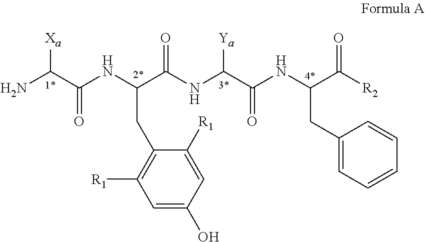

or a pharmaceutically acceptable salt, hydrate, solvate and/or tautomer thereof, wherein, each R₁ is independently H or —CH₃; R₂ is —OH or —NH₂; Xₐ and Yₐ are each independently selected from

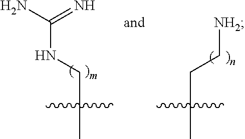

each m is 2, 3 or 4; each n is independently 1, 2, or 3; and the absolute stereochemistry at each of stereocenters 1*, 2*, 3*, and 4* is independently D or L.

For example, the peptide can be of formula A-1 (SEQ ID NO: 2):

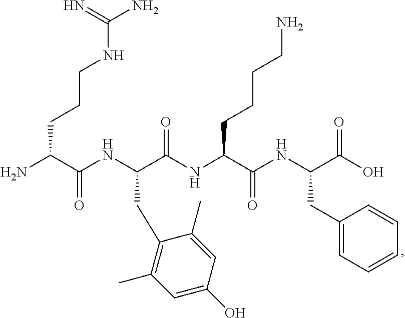 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof. The peptide can be of formula A-2 (SEQ ID NO: 3):

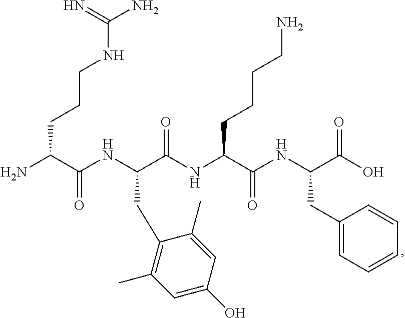 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof. The peptide can be of formula A-3 (SEQ ID NO: 4):

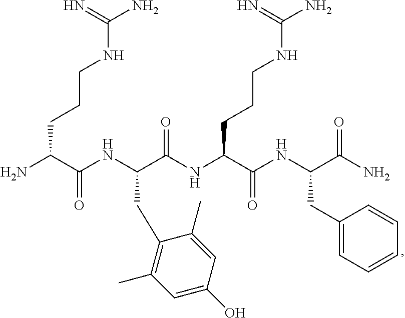 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O]

or a pharmaceutically acceptable salt, hydrate, solvate and/or tautomer thereof. The peptide can be of formula A-4 (SEQ ID NO: 5):

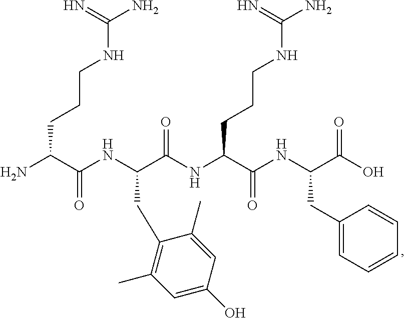 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O]

or a pharmaceutically acceptable salt, hydrate, solvate and/or tautomer thereof. The peptide can be of formula A-5:

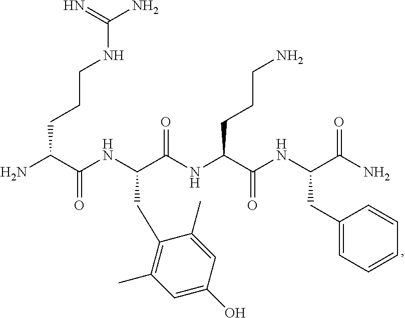 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O]

or a pharmaceutically acceptable salt, hydrate, solvate and/or tautomer thereof. The peptide can be of formula A-6:

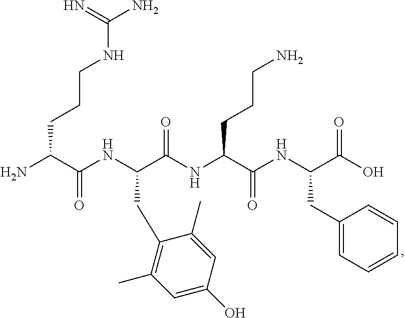 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

or a pharmaceutically acceptable salt, hydrate, solvate and/or tautomer thereof. The peptide can be of formula A-7 (SEQ ID NO: 6):

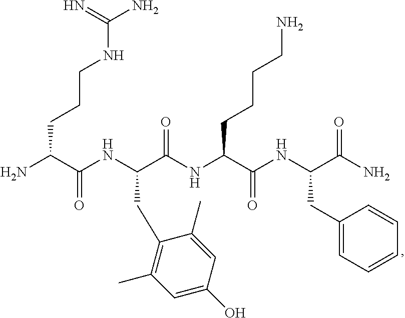 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof. The peptide can be of formula A-8 (SEQ ID NO: 7):

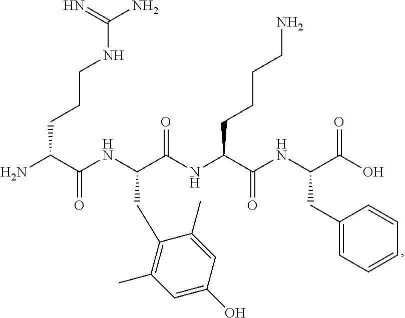 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof. The peptide can be administered individually or as a mixture comprising two or more of the peptides as defined herein. As noted, the peptide or mixture of peptides can be administered alone, in a formulation (e.g. medicament) or in combination with one or more other active ingredients. In some embodiments, the pharmaceutically acceptable salt can be selected from a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt.

The peptide or mixture of peptides and PMO(s) and/or PPMO(s), and/or other therapeutic agent(s)/drug(s) can be administered by any known or future developed mode of administration. For example, administration can be oral. Administration can be systemic. Administration can be subcutaneous. Administration can be intravenous. Administration can be topical, intraperitoneal, intradermal, transdermal, ophthalmical, retro-orbital, intrathecal, intracerebroventricular, iontophoretical, transmucosal, intravitreal, intranasal, or intramuscular. In some embodiments, peptide, mixture of peptides and/or the other therapeutic agent(s)/drug(s) are separately, sequentially or simultaneously administered. In some embodiments, administration of the peptide or mixture of peptides with another therapeutic agent produces a synergistic therapeutic effect.

In some embodiments, the peptide or mixture of peptides and PMO(s) and/or PPMO(s) are administered to the subject for 6 weeks or more. In some embodiments, the peptide or mixture of peptides and PMO(s) and/or PPMO(s) are administered to the subject for 12 weeks or more. In some embodiments, the peptide or mixture of peptides and PMO(s) and/or PPMO(s) are administered to the subject for 24 weeks or more. In some embodiments, the peptide or mixture of peptides and PMO(s) and/or PPMO(s) are administered to the subject for 48 weeks or more. In some embodiments, the peptide or mixture of peptides and PMO(s) and/or PPMO(s) are administered to the subject for 72 weeks or more. In some embodiments, the peptide or mixture of peptides and PMO(s) and/or PPMO(s) are administered to the subject for 96 weeks or more. In some embodiments, the peptide or mixture of peptides and PMO(s) and/or PPMO(s) are administered to the subject for 2 years or more. In some embodiments, the peptide or mixture of peptides and PMO(s) and/or PPMO(s) are administered to the subject for 3 years or more. In some embodiments, the peptide or mixture of peptides and PMO(s) and/or PPMO(s) are administered until no continued therapeutic benefit is observed. In some embodiments, the peptide or mixture of peptides and PMO(s) and/or PPMO(s) are administered until the end of life or near end of life of the subject. In some embodiments, the subject is a human and administration of the peptide or mixture of peptides and PMO(s) and/or PPMO(s) begins as soon as symptoms of DMD or BMD are diagnosed or observed and continues throughout the lifetime of the subject.

The peptide or mixture of peptides and PMO(s) and/or PPMO(s) can be administered at any reasonable interval. The interval of administration (i.e., dosing) will depend on several factors including the mode of administration, the dose to be administered, the formulation of the active ingredients, the toxicity of the formulation and any allergies, or other traits of the subject. Those of skill in the art will be able to determine the proper interval for dosing. In some embodiments, dosing will occur about once per day. In some embodiments, dosing will occur about twice per day. In some embodiments, dosing will occur about thrice per day. In some embodiments, dosing will occur about once every other day. In some embodiments, dosing will occur about once per week. In some embodiments, dosing will occur about once every other week. In some embodiments, dosing will occur about once per month. In some embodiments, dosing will occur about once every other month. In some embodiments, dosing will occur about once every three months. In some embodiments, dosing will occur about once every six months. In some embodiments, dosing will occur about once every nine months. In some embodiments, dosing will occur about once every year. In some embodiments, the interval of administration (i.e., dosing) of the peptide or mixture of peptides may differ from the interval of administration of the PMO(s) and/or PPMO(s). For example, in some embodiments, the peptide or mixture of peptides may be administered daily while the PMO(s) and/or PPMO(s) may be administered weekly. In some embodiments, the peptide or mixture of peptides and PMO(s) and/or PPMO(s) are administered as a depot formulation comprising a peptide or mixture of peptides and PMO(s) and/or PPMO(s) that are encapsulated by, or disposed within, silica microparticles.

In some embodiments, the methods described herein are directed to administration of the peptide or mixture of peptides described herein in combination with at least one PMO and/or PPMO. For example, in some embodiments, the methods described herein are directed to administration of the peptide or mixture of peptides in combination with a PMO such as Eteplirsen (Exondys 51®), Golodirsen (Vyondys 53™), Viltolarsen (Viltepso®), or Casimersen (Amondys 45™), or PPMO. In some embodiments, the peptide administered in combination with the PMO(s) or PPMO(s) is H-D-Arg-2′6′-Dmt-Lys-Phe-NH₂ (SEQ ID NO: 2) or its carboxylate form, H-D-Arg-2′6′-Dmt-Lys-Phe-OH (SEQ ID NO: 3) (or a pharmaceutically acceptable salt, hydrate, solvate and/or tautomer of either of the foregoing). Such combination of drugs may augment dystrophin expression in the subject as compared to a control subject administered the peptide or the PMO or PPMO alone. In some embodiments, the administration of the peptide augments PMO- or PPMO-mediated dystrophin expression. In some embodiments, the administration of the peptide in combination with one or more PMOs or PPMOs produces synergistic effects in the expression of dystrophin.

### Methods & Treatments:

In one aspect, the present disclosure provides methods for treating the signs, symptoms, or severity of muscular dystrophy in a mammalian subject suffering from DMD or BMD, comprising administering to a subject in need thereof a therapeutically effective amount of a therapeutically active peptide or mixture of peptides and one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) an ARB; and (iv) a beta blocker. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In another aspect, the present disclosure provides methods for inhibiting the signs, symptoms, or severity of muscular dystrophy in a mammalian subject suffering from DMD or BMD, comprising administering to the subject in need thereof a therapeutically effective amount of a therapeutically active peptide or mixture of peptides and one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; and (iii) a beta blocker. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In still another aspect, the present disclosure provides methods for preventing the signs, symptoms, or severity of muscular dystrophy in a mammalian subject suffering from DMD or BMD, comprising administering to the subject in need thereof a therapeutically effective amount of a therapeutically active peptide or mixture of peptides and one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) an ARB; and (iv) a beta blocker. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In yet another aspect, the present disclosure provides methods for ameliorating the signs, symptoms, or severity of muscular dystrophy in a mammalian subject suffering from DMD or BMD, comprising administering to the subject in need thereof a therapeutically effective amount of a therapeutically active peptide or mixture of peptides and one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) an ARB; and (iv) a beta blocker. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In still a further aspect, the present disclosure provides methods for delaying the onset of the signs, symptoms, or severity of muscular dystrophy in a mammalian subject suffering from DMD or BMD, comprising administering to the subject in need thereof a therapeutically effective amount of a therapeutically active peptide or mixture of peptides and one or more PMOs and/or PPMOs as described in more detail below, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) a beta blocker; and (iv) an ARB. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In yet another aspect, the present disclosure provides methods for delaying the onset of muscular dystrophy in a mammalian subject suspected of having or at risk for developing DMD or BMD, comprising administering to the subject in need thereof a therapeutically effective amount of a therapeutically active peptide or mixture of peptides and one or more PMOs and/or PPMOs as described in more detail below, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) a beta blocker; and (iv) an ARB. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In yet another aspect, the present disclosure provides methods for augmenting the production of dystrophin in a mammalian subject having, suspected of having or at risk for developing DMD or BMD, comprising administering to the subject in need thereof a therapeutically effective amount of a therapeutically active peptide or mixture of peptides and one or more PMOs and/or PPMOs as described in more detail below, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) in combination with one or more of the following additional therapeutic agents: (i) a corticosteroid; (ii) an ACE inhibitor; (iii) a beta blocker; and (iv) an ARB. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

A mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a non-human primate. In some embodiments, the mammal is a human.

Said peptide or mixture of peptides and one or more PMOs and/or PPMOs can be administered alone, in a composition or formulation or in combination with one or more additional therapeutic agents. In some embodiments, the compositions are used as medicaments or in the preparation of medicaments for: (i) treating the signs, symptoms, or severity of muscular dystrophy in a mammalian subject suffering from DMD or BMD; (ii) inhibiting the signs, symptoms, or severity of muscular dystrophy in a mammalian subject suffering from DMD or BMD; (iii) preventing the signs, symptoms, or severity of muscular dystrophy in a mammalian subject suffering from DMD or BMD; (iv) ameliorating the signs, symptoms, or severity of muscular dystrophy in a mammalian subject suffering from DMD or BMD; (v) delaying the onset of the signs, symptoms, or severity of muscular dystrophy in a mammalian subject suspected of having or at risk for developing DMD or BMD, (vi) delaying the onset of muscular dystrophy in a mammalian subject having, suspected of having or at risk for developing DMD or BMD, or (vii) augmenting the production of dystrophin in a mammalian subject having, suspected of having or at risk for developing DMD or BMD. In some embodiments, the compositions are used as medicaments or in the preparation of medicaments for augmenting dystrophin expression in a subject as compared to an untreated subject or as compared to a control subject administered either the peptide or peptide mixture alone or the PMO(s) and/or PPMO(s) alone. In some embodiments, the subject is a human.

In some embodiments, the peptide or mixture of peptides and one or more PMOs and/or PPMOs is/are administered in a depot formulation (discussed below), such as a silica-based depot formulation, wherein the peptide or peptides and one or more PMOs and/or PPMOs are encapsulated/encased in silica particles (nanoparticles or microparticles) that slowly release the peptide or peptides and one or more PMOs and/or PPMOs over time (e.g. by sustained and/or controlled release over days, weeks or months). For example, the depot formulation of the peptides and one or more PMOs and/or PPMOs may be injected subcutaneously to provide for long-term systemic release of the peptide or peptides and one or more PMOs and/or PPMOs to the subject.

Administration of the peptide (or mixture of peptides) and one or more PMOs and/or PPMOs, or a composition comprising the peptide(s) and one or more PMOs and/or PPMOs may exhibit various beneficial effects on dystrophin expression in the subject to which the peptide (or mixture of peptides) and one or more PMOs and/or PPMOs or composition is administered. For example, administration of the peptide (or mixture of peptides) and one or more PMOs and/or PPMOs or composition may increase dystrophin expression in the subject and/or increase expression of functional dystrophin protein in the subject as compared to an untreated subject (or untreated control group of subjects) or as compared to a subject (or control group of subjects) administered the PMOs and/or PPMOs alone or the peptide (or mixture of peptides) alone. Administration of the peptide (or mixture of peptides) and one or more PMOs and/or PPMOs or composition may increase dystrophin expression in skeletal, cardiac, and/or smooth muscle in the subject as compared to an untreated subject (or untreated control group of subjects) or as compared to a subject (or control group of subjects) administered the PMOs and/or PPMOs alone or the peptide (or mixture of peptides) alone. Administration of the peptide (or mixture of peptides) and one or more PMOs and/or PPMOs or composition may decrease inflammation in skeletal, cardiac, and/or smooth muscle in the subject as compared to an untreated subject (or untreated control group of subjects) or as compared to a subject (or control group of subjects) administered the PMOs and/or PPMOs alone or the peptide (or mixture of peptides) alone.

### Peptides & Mixtures of Peptides:

The aforementioned methods can be practiced with peptides or mixtures of peptides. Peptides suitable for use in the aforementioned methods are peptides of generic Formula A or a pharmaceutically acceptable salt, hydrate, solvate and/or tautomer thereof, wherein Formula A is:

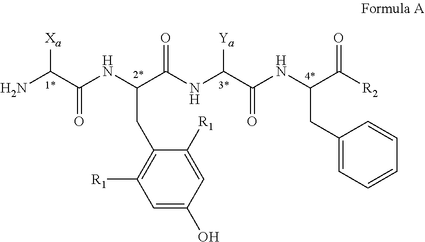

wherein, each R₁ is independently H or —CH₃; R₂ is —OH or —NH₂; Xₐ and Yₐ are each independently selected from

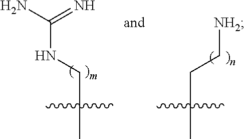

each m is 2, 3 or 4; each n is independently 1, 2, or 3; and the absolute stereochemistry at each of stereocenters 1*, 2*, 3* and 4* is independently D or L.

In some embodiments, the peptide of generic Formula A is a peptide of formula A-1:

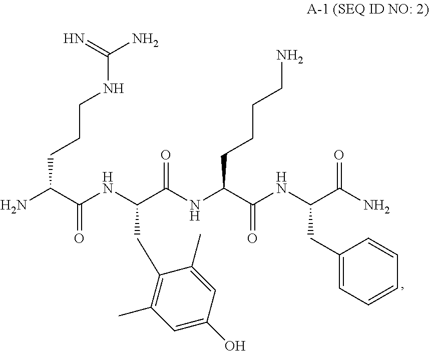 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

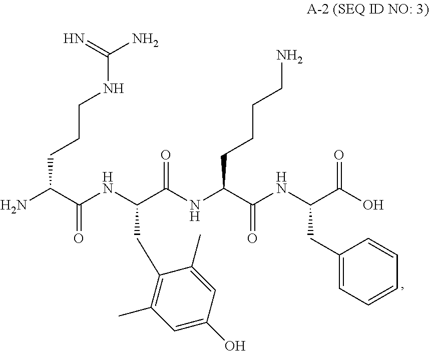 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-3:

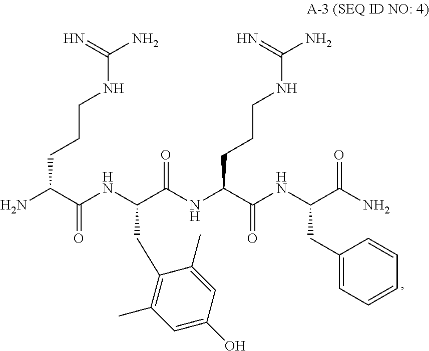 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-4:

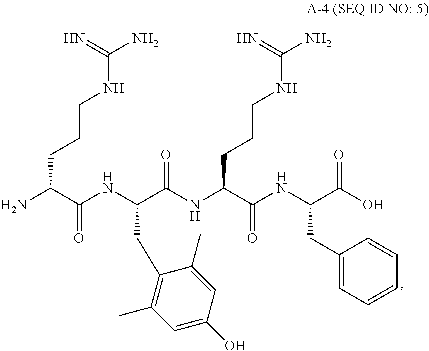 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-5:

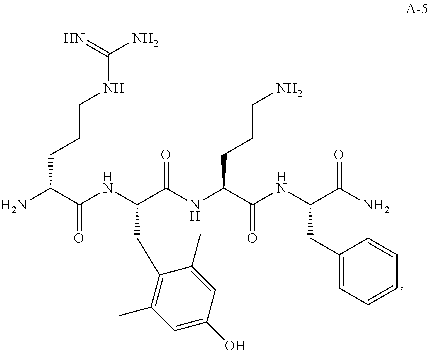 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-6:

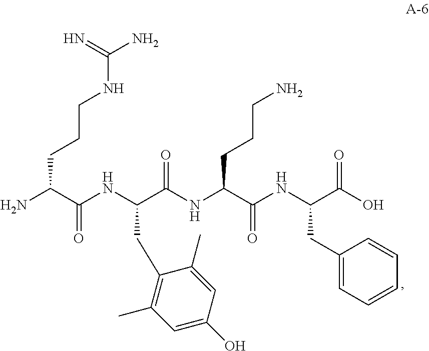 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-7:

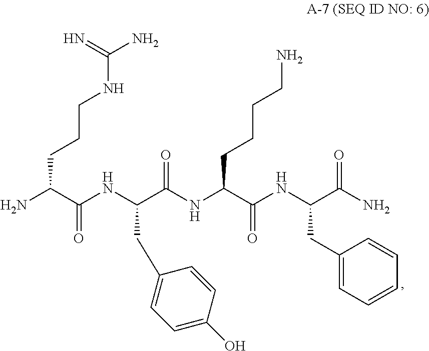 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-8:

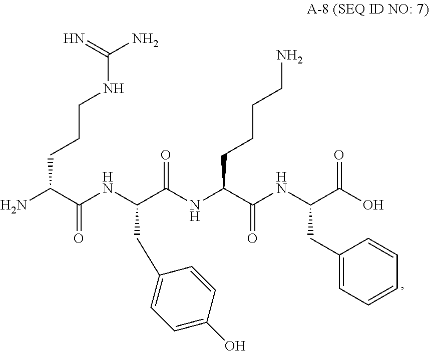 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, mixtures of two or more of the above described peptides are used as a/the therapeutic agent. Such mixtures may be present intentionally (e.g., by mixing the peptides post synthesis) or fortuitously (e.g., by the hydrolysis of a C-terminal amide to a C-terminal carboxylic acid). Whenever reference is made herein to a ‘peptide or mixture of peptides’, it is implied and intended that each individual peptide of said “peptide or mixture of peptides” can exist as a free acid/base, in zwitterionic form or in any salt form, including in a pharmaceutically acceptable salt form (e.g., See: FIGS. 4 and 5 for various salt forms of A-1 & A-2).

### Peptide Synthesis:

The peptides may be synthesized by any of the methods well known in the art. The peptides can be prepared using solid-phase synthesis methodology. The peptides can be synthesized by using solution-phase methodology. Suitable methods for chemically synthesizing the peptides include, for example, those described in any of WO 2004/070054, WO 2018/03490, WO 2019/099481, or WO 2018/187400. In some embodiments, the peptides are C-terminal amides and in some embodiments the peptide are C-terminal carboxylic acids. Peptides that are C-terminal amides can be converted to peptides comprising C-terminal acids by simple hydrolysis as described in Example 3, below.

For example, the peptides disclosed herein can be prepared using any peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132; Stuart and Young in Solid Phase Peptide Synthesis, Second Edition, Pierce Chemical Company (1984), and in Methods Enzymol., 289, Academic Press, Inc., New York (1997)). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. Typically in solid phase synthesis, after coupling, a capping step is performed to cap (render unreactive) any unreacted amine groups. These steps (i.e., deprotection, coupling, and optionally capping) may be repeated to form the desired peptide chain. Once the desired peptide chain is complete, the peptide may be cleaved from the solid support.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z, cbz or Cbz), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase or solution-phase peptide synthesis discussed herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), and its HCl salt (EDC-HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include (1-[Bis(dimethylamino)methylene]-1H-1,2,3-triazolo[4,5-b]pyridinium 3-oxide hexafluorophosphate (HATU) and (2-(1H-benzotriazol-1-yl)-1,1,3,3-tetramethyluronium hexafluorophosphate (HBTU), generally used in combination with an organic base such as N,N-diisopropylethylamine (DIEA) and a hindered pyridine-type base such as lutidine or collidine.

In some embodiments, the amino acids can be activated toward coupling by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996); and WO 2018/034901. Such methods of peptide synthesis may be used to produce the peptides disclosed herein either by solution-phase or solid-phase methodology.

Salt Forms (Some of which are Pharmaceutically Acceptable Salts) & Other Forms:

Compounds of Formula A (including without limitation A-1, A-2, A-3, A-4, A-5, A-6, A-7 and A-8) can exist in various forms, such as in salt form(s) (such as a pharmaceutically acceptable salt form), in tautomeric form(s), in solvated form(s) and/or in hydrate form(s).

For example, FIG. 4 illustrates various forms that Compound A-2 can take and FIG. 5 illustrates various forms that Compound A-1 can take. With reference to FIG. 4, (20) illustrates a mono-basic salt form of Compound A-2, wherein the C-terminal carboxylate has been ionized as its base-salt. As illustrated, the basic generic salt represented by YOH can ionize to produce Y+ and OH− and thereby ionize Compound A-2 (21) to form (20). The generic basic salt represented by YOH could be, for example, sodium hydroxide (NaOH), potassium hydroxide (KOH) or lithium hydroxide (LiOH). The mono-basic salt form (20) can be protonated with acid to form Compound A-2 (21). However, Compound A-2 (21) can also be represented in zwitterionic form (22) resulting from the internal distribution of a proton between the carboxylate and one of the basic groups. Compound A-2 ((21) or (22)) can be further protonated with a single equivalent of acid (e.g., represented by HX wherein H+ is the proton and X− represents the counterion and is embodied by acids such as HCl, HBr or HI) to thereby produce a mono-acid salt (23). The mono-acid salt (23) can be further acidified with another equivalent of acid to thereby produce a bis-acid salt (24). The bis-acid salt (24) can be further acidified with another equivalent of acid to thereby produce a tris-acid salt (25). One of skill in the art will appreciate that these transitions between the various salt forms are easily accomplished by use of an appropriate amount of acid or base. One of skill in the art will further appreciate that such transitions between salt forms are also applicable to any compounds represented by Formula A, including without limitation Compounds A-1, A-3, A-4, A-5, A-6, A-7 or A-8.

The peptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the pharmaceutically acceptable salt is a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt.

Certain compound(s)/peptide(s) disclosed in the present disclosure can exist in unsolvated forms as well as solvated forms, including hydrated forms. Solvated forms can exist, for example, because it is difficult or impossible to remove all the solvent from the peptide post synthesis. In general, the solvated forms are equivalent to unsolvated forms and are encompassed within the scope of the present disclosure.

Certain compound(s)/peptide(s) of the present disclosure may exist in crystalline form, multiple crystalline forms, amorphous forms or any combination of the foregoing. Certain compound(s)/peptide(s) of the present disclosure may exist in various tautomeric forms. Certain compound(s)/peptide(s) of the present disclosure may exist in various salt forms or mixtures of salt forms. In general, all physical forms of the compound(s)/peptide(s) disclosed herein are equivalent for the uses contemplated by the present disclosure and are intended to be within the scope of the present disclosure.

### Chiral/Stereochemistry Considerations:

Peptides/compounds described herein can comprise one or more asymmetric centers, and thus can exist in various isomeric forms, e.g., enantiomers and/or diastereomers (i.e., stereoisomers). Chiral centers in illustrated structures (including the claims) may be identified herein by use of an asterisk (*). For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure of the present application additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess); as purity is a relative term in the sense that it is exceedingly difficult to achieve 100% purity. In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that for a “D”-amino acid the configuration is “R” and for an “L”-amino acid, the configuration is “S”. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g., as compared with the other enantiomer). In certain embodiments, the weights are based upon total weight of all enantiomers or stereoisomers of the compound.

In the compositions provided herein, an enantiomerically pure compound (e.g., a peptide) can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the enantiomers of the compound. In certain embodiments, the active ingredient can be formulated with little or no excipient or carrier.

### Compositions, Formulations & Dosing:

This disclosure further relates to compositions that can be used in the disclosed methods wherein the composition comprises at least one peptide of Formula A (e.g., Compound A-1, A-2, A-3, A-4, A-5, A-6, A-7 or A-8), and at least one PMO and/or PPMO, but may also include or more of the following compounds/therapeutic agents: (i) a corticosteroid; (ii) an ACE inhibitor; (iii) a beta blocker; and (iv) an ARB. Such a composition can be formed, for example, by dissolving or suspending the selected compound(s)/peptide (or mixture of peptides) and PMO(s) and/or PPMO(s) in water, buffer, detergent, excipient, organic solvent or a mixture of two or more of the foregoing. In some embodiments, the composition can be prepared by dissolving or suspending the selected compound(s)/peptide(s) and PMO(s) and/or PPMO(s) in water. In some embodiments, the composition can be prepared by dissolving or suspending the selected compound(s)/peptide(s) and PMO(s) and/or PPMO(s) in buffer. In some embodiments, the composition can be prepared by dissolving or suspending the selected compound(s)/peptide(s) and PMO(s) and/or PPMO(s) in excipient. In some embodiments, the composition can be prepared by dissolving or suspending the selected compound(s)/peptide(s) and PMO(s) and/or PPMO(s) in a pharmaceutically acceptable carrier. In some embodiments, the composition or formulation is a medicament.

The peptide or mixture of peptides and PMO(s) and/or PPMO(s) and optionally other therapeutic agents/drugs may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, phosphoric, monohydrogenphosphoric, dihydrogenphosphoric, sulfuric, monohydrogensulfuric, hydriodic, carbonic, monohydrogencarbonic, or phosphorous acids and the like, as well as the salts derived from organic acids like acetic, propionic, isobutyric, maleic, malonic, benzoic, succinic, suberic, fumaric, lactic, mandelic, oxalic, phthalic, benzenesulfonic, p-toluenesulfonic, citric, tartaric, methanesulfonic, trifluoroacetic, and the like. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present disclosure may contain both basic and acidic functionalities that allow the compounds to be converted into either base or acid addition salts (see, e.g., FIG. 4, FIG. 5). These salts may be prepared by methods known to those skilled in the art. Other pharmaceutically acceptable carriers known to those of skill in the art are suitable for use with the present technology. Suitable buffering agents may include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In some embodiments, the compositions or formulations can be used as medicaments or in the preparation of medicaments for: (i) treating the signs, symptoms, or severity of muscular dystrophy in a mammalian subject suffering from DMD or BMD; (ii) inhibiting the signs, symptoms, or severity of muscular dystrophy in a mammalian subject suffering from DMD or BMD; (iii) preventing the signs, symptoms, or severity of muscular dystrophy in a mammalian subject suffering from DMD or BMD; (iv) ameliorating the signs, symptoms, or severity of muscular dystrophy in a mammalian subject suffering from DMD or BMD; (v) delaying the onset of the signs, symptoms, or severity of muscular dystrophy in a mammalian subject suspected of having or at risk for developing DMD or BMD; (vi) delaying the onset of muscular dystrophy in a mammalian subject having, suspected of having or at risk for developing DMD or BMD; or (vii) augmenting the production of dystrophin in a mammalian subject having, suspected of having or at risk for developing DMD or BMD. In some embodiments, the compositions are used as medicaments or in the preparation of medicaments for augmenting dystrophin expression in a subject as compared to an untreated subject or as compared to a control subject administered either the peptide or peptide mixture alone or the PMO(s) and/or PPMO(s) alone.

The compositions and methods of the present disclosure may be utilized to treat an individual/subject in need thereof. In certain embodiments, the individual is a mammal such as a human, or a non-human mammal. When administered to an animal, such as a human, the composition or the compound/peptide and PMO(s) and/or PPMO(s) is preferably administered as a pharmaceutical composition comprising, for example, a peptide or mixture of peptides and PMO(s) and/or PPMO(s) and an excipient or pharmaceutically acceptable carrier.

As stated above, an “effective amount” refers to any amount of the active compound (e.g., a peptide or mixture of peptides and PMO(s) and/or PPMO(s); alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease or condition being treated, the particular compound of the present application being administered, the size of the subject, or the severity of the disease or condition. The effective amount may be determined during pre-clinical trials and/or clinical trials by methods familiar to physicians and clinicians. One of ordinary skill in the art can empirically determine the effective amount of a particular peptide or mixture of peptides of the present application and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g., a pump).

For any compound (e.g., a peptide or mixture of peptides) described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Peptides/compounds and PMO(s) and/or PPMO(s) (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, any of the animal model system known in the art can be used prior to administration to human subjects.

Dosage, toxicity and therapeutic efficacy of any therapeutic peptides, PMO(s), PPMO(s), compounds, compositions (e.g., formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography, optionally coupled with mass spectroscopy detection (e.g. LC/MS).

The effective amount may be determined during pre-clinical trials and clinical trials by methods familiar to physicians and clinicians. An effective amount of the compound(s)/peptide(s) and PMO(s) and/or PPMO(s) useful in the methods disclosed herein may be administered to a mammal in need thereof by any of a number of well-known methods for administering pharmaceutical compounds. The peptide and PMO(s) and/or PPMO(s) may be administered systemically or locally.

Typically, an effective amount of the compound(s)/peptide(s) and PMO(s) and/or PPMO(s), sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 500 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of a peptide, PMO, or PPMO can range from 0.001-10,000 micrograms per kg body weight. In one embodiment, peptide, PMO, or PPMO concentrations in a carrier range from 0.2 to 2000 micrograms per delivered milliliter. An exemplary treatment regimen entails administration of the peptide and/or PMO or the peptide and/or PPMO once per day or once a week.

In some embodiments, a therapeutically effective amount of peptide and PMO and/or PPMO may be defined as a concentration of peptide and PMO and/or PPMO at the target tissue (e.g., skeletal muscle tissue) of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

In certain embodiments, intravenous administration of a compound (e.g., a peptide or mixture of peptides, PMO(s), or PPMO(s)) may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

In certain embodiments, subcutaneous administration of a compound (e.g. a peptide or mixture of peptides, PMO(s), or PPMO(s)) may typically be from 0.1 mg/kg/day to 20 mg/kg/day. In one embodiment, subcutaneous administration of a compound may typically be from 0.1 mg/kg/day to 2 mg/kg/day. In one embodiment, subcutaneous administration of a compound may typically be from 0.5 mg/kg/day to 5 mg/kg/day. In one embodiment, subcutaneous administration of a compound may typically be from 1 mg/kg/day to 20 mg/kg/day. In one embodiment, subcutaneous administration of a compound may typically be from 1 mg/kg/day to 10 mg/kg/day. In one embodiment, subcutaneous administration of a compound may typically be from 0.5 mg/kg/day to 1.0 mg/kg/day. In some embodiments, subcutaneous administration of a compound may typically be 10 mg/kg/day. In some embodiments, subcutaneous administration of a compound may typically be 9 mg/kg/day. In some embodiments, subcutaneous administration of a compound may typically be 8 mg/kg/day. In some embodiments, subcutaneous administration of a compound may typically be 7 mg/kg/day. In some embodiments, subcutaneous administration of a compound may typically be 6 mg/kg/day. In some embodiments, subcutaneous administration of a compound may typically be 5 mg/kg/day. In some embodiments, subcutaneous administration of a compound may typically be 4 mg/kg/day. In some embodiments, subcutaneous administration of a compound may typically be 3 mg/kg/day. In some embodiments, subcutaneous administration of a compound may typically be 2 mg/kg/day. In some embodiments, subcutaneous administration of a compound may typically be 1 mg/kg/day. In some embodiments, subcutaneous administration of a compound may typically be 0.9 mg/kg/day. In some embodiments, subcutaneous administration of a compound may typically be 0.8 mg/kg/day. In some embodiments, subcutaneous administration of a compound may typically be 0.75 mg/kg/day. In some embodiments, subcutaneous administration of a compound may typically be 0.7 mg/kg/day. In some embodiments, subcutaneous administration of a compound may typically be 0.6 mg/kg/day. In some embodiments, subcutaneous administration of a compound may typically be 0.5 mg/kg/day. In some embodiments, subcutaneous administration of a compound may typically be 0.4 mg/kg/day. In some embodiments, subcutaneous administration of a compound may typically be 0.3 mg/kg/day. In some embodiments, subcutaneous administration of a compound may typically be 0.25 mg/kg/day. In some embodiments, subcutaneous administration of a compound may typically be 0.2 mg/kg/day. In some embodiments, subcutaneous administration of a compound may typically be 0.1 mg/kg/day. Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous dose per day administration would be from one order to several orders of magnitude lower. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

The skilled artisan will appreciate that certain factors may influence the dosage, mode of administration and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The peptide, mixture of peptides, PMO(s), PPMO(s), or other therapeutic agent(s)/drug(s) can be administered by any known or future developed mode of administration. For example, administration can be oral. Administration can be systemic. Administration can be subcutaneous. Administration can be intravenous. Administration can be topical, intraperitoneal, intradermal, transdermal, ophthalmical, retro-orbital, intrathecal, intracerebroventricular, iontophoretical, transmucosal, intravitreal, intranasal, or intramuscular. In some embodiments, peptide or mixture of peptides and the other therapeutic agent(s)/drug(s) are separately, sequentially or simultaneously administered. In some embodiments, administration of the peptide or mixture of peptides in combination with PMO(s) and/or PPMO(s) produces a synergistic effect. In some embodiments, administration of the peptide or mixture of peptides in combination with PMO(s) and/or PPMO(s) with another therapeutic agent produces a synergistic therapeutic effect.

In some embodiments, the peptide or mixture of peptides and PMO(s) and/or PPMO(s) are administered to the subject for 6 weeks or more. In some embodiments, the peptide or mixture of peptides and PMO(s) and/or PPMO(s) are administered to the subject for 12 weeks or more. In some embodiments, the peptide or mixture of peptides and PMO(s) and/or PPMO(s) are administered to the subject for 24 weeks or more. In some embodiments, the peptide or mixture of peptides and PMO(s) and/or PPMO(s) are administered to the subject for 48 weeks or more. In some embodiments, the peptide or mixture of peptides and PMO(s) and/or PPMO(s) are administered to the subject for 72 weeks or more. In some embodiments, the peptide or mixture of peptides and PMO(s) and/or PPMO(s) are administered to the subject for 96 weeks or more. In some embodiments, the peptide or mixture of peptides and PMO(s) and/or PPMO(s) are administered to the subject for 2 years or more. In some embodiments, the peptide or mixture of peptides and PMO(s) and/or PPMO(s) are administered to the subject for 3 years or more. In some embodiments, the peptide or mixture of peptides and PMO(s) and/or PPMO(s) are administered until no continued therapeutic benefit is observed. In some embodiments, the peptide or mixture of peptides is administered until the end of life of the subject.

The peptide or mixture of peptides and PMO(s) and/or PPMO(s) can be administered at any reasonable interval. The interval of administration (i.e., dosing) will depend on several factors including the mode of administration, the dose to be administered, the formulation of the active ingredients, the toxicity of the formulation and any allergies or other traits of the subject. Those of skill in the art will be able to determine the proper interval for dosing. In some embodiments, dosing will occur about once per day. In some embodiments, dosing will occur about twice per day. In some embodiments, dosing will occur about thrice per day. In some embodiments, dosing will occur about once every other day. In some embodiments, dosing will occur about once per week. In some embodiments, dosing will occur about once every other week. In some embodiments, dosing will occur about once per month. In some embodiments, dosing will occur about once every other month. In some embodiments, dosing will occur about once every three months. In some embodiments, dosing will occur about once every six months. In some embodiments, dosing will occur about once every nine months. In some embodiments, dosing will occur about once every year.

The pharmaceutical compositions (e.g., a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 2, 3, 4, 5, 6, 7 days, weeks, months or more of treatment).

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound (e.g., a peptide or mixture of peptides, PMO(s), or PPMO(s)) of the present application can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and must be preserved against the contaminating action of microorganisms such as bacteria and fungi.

Sterile injectable solutions (e.g., a formulation or medicament) can be prepared by incorporating the active compound (e.g., a peptide or mixture of peptides, PMO(s), or PPMO(s)) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

The therapeutic compounds (e.g., a peptide or mixture of peptides, PMO(s), or PPMO(s)) or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include aqueous solutions of the active compounds (e.g., a peptide or mixture of peptides, PMO(s), PPMO(s)) in water-soluble form. Additionally, suspensions of the therapeutic compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the therapeutic compounds to allow for the preparation of highly concentrated solutions.

For oral administration, the compounds (e.g., a peptide or mixture of peptides, PMO(s), PPMO(s)) can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above may be chemically modified so that oral delivery of the derivative is efficacious. Generally, the chemical modification contemplated is the attachment of at least one moiety to the therapeutic agent(s), ingredient(s), and/or excipient(s), where said moiety permits: (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound (e.g., a peptide or mixture of peptides, PMO(s), PPMO(s)) or pharmaceutical composition can be included in the formulation as fine multi-particulates in the form of granules or pellets of particle size about 1-2 mm. The formulation of the material for capsule administration could also be as a powder, lightly compressed plugs or even as tablets. The therapeutic compound or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound or pharmaceutical composition with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of the therapeutic compound (e.g., a peptide or mixture of peptides, PMO(s), PPMO(s)) or composition into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The compounds, peptides, peptide mixtures, PMO(s), PPMO(s), and compositions disclosed herein can be included in a formulation as fine multi-particulates in the form of granules or pellets of particle size about 1 mm. The formulation of the material for capsule administration could also be as a powder, lightly compressed plugs or even as tablets. The formulation could be prepared by compression.

For buccal administration, the compositions may take the form of tablets or lozenges formulated in conventional manner.

For topical administration, a compound, peptide or mixture of peptides, PMO(s), or PPMO(s) may be formulated as solutions, gels, ointments, creams, suspensions, etc., as are well-known in the art.

For administration by inhalation, peptides, PMO(s), PPMO(s), compounds or compositions (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound and a suitable powder base such as lactose or starch.

Nasal delivery of a therapeutic compound (e.g. a peptide or mixture of peptides, PMO(s), PPMO(s)) or pharmaceutical composition of the present application is also contemplated. Nasal delivery allows the passage of a therapeutic compound or pharmaceutical composition of the present application to the blood stream directly after administering the therapeutic compound or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound or pharmaceutical composition.

Alternatively, the therapeutic compound (e.g., a peptide or mixture of peptides, PMO(s), PPMO(s)) or pharmaceutical composition may be in powder form for constitution with a suitable vehicle, e.g., sterile pyrogen-free water, before use.

Also contemplated herein is pulmonary delivery of the compounds, peptide or mixture of peptides, PMO(s), or PPMO(s) disclosed herein (or salts, hydrates, solvates and/or tautomers thereof). The compound, peptide or mixture of peptides, PMO(s), or PPMO(s) are delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the compound, peptides or mixtures of peptides, PMO(s), or PPMO(s) disclosed herein. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro. Chemically modified compound may also be prepared in different formulations depending on the type of chemical modification or the type of device employed.

Formulations suitable for use with a nebulizer, either jet or ultrasonic, will typically comprise a peptide or mixture of peptides disclosed herein dissolved in water at a concentration of about 0.1 to 25 mg of biologically active compound (e.g., a peptide or mixture of peptides, PMO(s), PPMO(s)) per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the peptide or mixture of peptides, PMO(s), PPMO(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a peptide or mixture of peptides, PMO(s), or PPMO(s) disclosed herein and may also include a bulking agent, such as lactose, sorbitol, sucrose, trehalose, or mannitol in amounts which facilitate dispersal of the powder from the device, e.g., 50 to 90% by weight of the formulation. The compound (or derivative) should advantageously be prepared in particulate form with an average particle size of less than 10 micrometers (m), most preferably 0.5 to 5 m, for most effective delivery to the deep lung.

In addition to the formulations described above, a peptide or mixture of peptides, PMO(s), or PPMO(s) may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The peptides or mixture of peptides, PMO(s), or PPMO(s) may be provided in particles. Particles as used herein means nanoparticles or microparticles/microspheres (or in some instances larger particles) which can consist in whole or in part of the compound or the other therapeutic agent(s) as described herein. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The peptides or mixture of peptides, PMO(s), or PPMO(s) also may be dispersed throughout the particles. The peptides or mixture of peptides, PMO(s), or PPMO(s) also may be adsorbed into the particles. The particles may be of any order release kinetics, including zero-order release, first-order release, second-order release, delayed release, sustained release, immediate release, and any combination thereof, etc. The particle may include, in addition to the peptides or mixture of peptides, PMO(s), or PPMO(s), any of those materials routinely used in the art of pharmacy and medicine, including, but not limited to, erodible, nonerodable, biodegradable, or nonbiodegradable material or combinations thereof. The particles may be microcapsules which contain the compound in a solution or in a semi-solid state. The particles may be of virtually any shape.

Both non-biodegradable and biodegradable polymeric materials can be used in the manufacture of particles for delivering the peptides or mixture of peptides, PMO(s), or PPMO(s). Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and polycaprolactone.

A therapeutic compound (e.g., a peptide or mixture of peptides, PMO(s), PPMO(s)) or other therapeutic agent or mixtures thereof can be formulated in a carrier system. The carrier can be a colloidal system. The carrier or colloidal system can be a liposome, a phospholipid bilayer vehicle. In one embodiment, therapeutic compound or other therapeutic agent or mixtures thereof can be encapsulated in a liposome while maintaining integrity of the therapeutic compound or other therapeutic agent or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g., a peptide or mixture of peptides, PMO(s), PPMO(s)) or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or poly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compound (e.g., a peptide or mixture of peptides, PMO(s), PPMO(s)) or other therapeutic agent or mixtures thereof are prepared with carriers that will protect the therapeutic compound, other therapeutic agent or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient for at least 2 days. In some embodiments, the implant or depot formulation is constructed and arranged to deliver therapeutic or prophylactic levels of the active ingredient for at least 7 days. In some embodiments, the implant or depot formulation is constructed and arranged to deliver therapeutic or prophylactic levels of the active ingredient for at least 14 days. In some embodiments, the implant or depot formulation is constructed and arranged to deliver therapeutic or prophylactic levels of the active ingredient for at least 30 days. In some embodiments, the implant or depot formulation is constructed and arranged to deliver therapeutic or prophylactic levels of the active ingredient for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient for at least 180 days. In some embodiments, the implant or depot formulation is constructed and arranged to deliver therapeutic or prophylactic levels of the active ingredient for at least one year. In some embodiments, the implant or depot formulation is constructed and arranged to deliver therapeutic or prophylactic levels of the active ingredient for 15-30 days. In some embodiments, the implant or depot formulation is constructed and arranged to deliver therapeutic or prophylactic levels of the active ingredient for 30-60 days. In some embodiments, the implant or depot formulation is constructed and arranged to deliver therapeutic or prophylactic levels of the active ingredient for 60-90 days. In some embodiments, the implant or depot formulation is constructed and arranged to deliver therapeutic or prophylactic levels of the active ingredient for 90-120 days. In some embodiments, the implant or depot formulation is constructed and arranged to deliver therapeutic or prophylactic levels of the active ingredient for 120-180 days. In some embodiments, the long-term sustained release implants or depot formulation are well-known to those of ordinary skill in the art and include some of the release systems described above.

In some embodiments, such implants or depot formulation can be administered surgically. In some embodiments, such implants or depot formulation can be administered topically or by injection.

In some embodiments, the depot formulation comprises the peptide, or mixture of peptides, PMO(s), or PPMO(s) encapsulated or otherwise disposed within silica microparticles such those described in WO2000/050349, WO2001/013924, WO2001/015751, WO2001/040556, WO2002/080977, WO2005/082781, WO2007/135224, WO2008/104635, WO2014/207304 and WO2017/068845, wherein the active pharmaceutical ingredient to be delivered is the peptide or mixture of peptides, PMO(s), or PPMO(s) disclosed herein. In some embodiments, the depot formulation is a sustained release formulation such that it provides for gradual release of a peptide or peptides (e.g. the peptide of Formula A), PMO(s), or PPMO(s) over an extended period of time, and that preferably, although not necessarily, results in substantially constant blood levels of a drug over an extended time period. In some embodiments, the sustained release occurs over days, weeks or months. In some embodiments, the sustained release occurs over a month or months, such as 1-2 months, 2-4 months, 3-5 months, 3-6 months, 5-7 months, 6-8 months, 6-9 months or 8-12 months.

It will be understood by one of ordinary skill in the relevant arts that other suitable modifications and adaptations to the compositions and methods described herein are readily apparent from the description of the present technology contained herein in view of information known to the ordinarily skilled artisan, and may be made without departing from the scope of the present application or any embodiment thereof.

### Determination of the Biological Effect of the Peptides or Mixtures of Peptides:

In various embodiments, suitable in vitro or in vivo assays are performed to determine the effect of a peptide or mixture of peptides and PMO(s) and/or PPMO(s) and whether their administration is indicated for treatment. In various embodiments, in vitro assays can be performed with representative animal models, to determine if a given peptide or mixture of peptides and PMO(s) and/or PPMO(s) exerts the desired effect on the disease or, in some embodiments, the expression of dystrophin in the subject, such as in the skeletal, smooth and/or cardiac muscle(s). Compounds (e.g., a peptide or mixture of peptides, PMO(s), PPMO(s)) for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Animal models of DMD are known in the art, including, for example Golden retriever muscular dystrophy (GRMD) dogs, CXMDJ beagle dogs, hypertrophic feline muscular dystrophy (hfmd) cats, and mdx mice. See: Spurney C., Muscle Nerve 44(1):8-19 (2011); Willmann R. et al., Neuromuscular Disorders 19:241-249 (2009); Partridge T A, FEBS J. 280(17):4177-86 (2013) and Coley et al., “Effect of genetic background on the dystrophic phenotype in mdx mice”, Human Molecular Genetics, 2016, Vol. 25, No. 1, 130-145. More recently a rabbit model has been created that exhibits a very similar cardiac pathology to that observed in humans. See: Sui, T, et al., “A novel rabbit model of Duchenne muscular dystrophy generated by CRISPR/Cas9”, Disease Models & Mechanisms (2018) 11, dmm032201. Such models may be used to demonstrate the biological effect of the peptides and mixtures of peptides and PMO(s) and/or PPMO(s) disclosed herein on the onset, incidence, severity and progression of muscular dystrophy (including DMD and BMD) in subjects, including humans.

### Combination Therapy

In some embodiments, the peptide or mixtures of peptides and PMO(s) and/or PPMO(s) disclosed herein, may be combined with one or more additional therapies related to the treatment of (including without limitation the inhibition of, prevention of, amelioration of, or delaying the onset of) signs, symptoms, or severity of DMD or BMD in a subject, including a human subject. Additional therapeutic agents include, but are not limited to, corticosteroids, ACE inhibitors, ARB(s), beta-blockers, diuretics, angiotensin receptor blockers (ARBs), and idebenone. In some embodiments, the PMOs comprise Exondys 51® (Eteplirsen), Golodirsen (Vyondys 53™), Viltolarsen (Viltepso®), or Casimersen (Amondys 45™), or a PPMO. In some embodiments, the PMO comprises an appropriately designed exon-skipping oligomer that is relevant to the dystrophin lesion in a subject in need thereof, wherein the subject has been diagnosed with or is suspected of having a muscular dystrophy, such as DMD or BMD. By way of example, but not by limitation, the PMO comprises an antisense oligomer of about 20-50 nucleotides in length, or a pharmaceutically acceptable salt thereof, capable of binding a selected target in human dystrophin pre-mRNA to induce exon skipping in the human dystrophin gene, wherein the antisense oligomer comprises a sequence of bases that specifically hybridizes to a dystrophin exon target region.

In some embodiments, the corticosteroids are selected from the group consisting of prednisone and deflazacort. In some embodiments, the ACE inhibitors are selected from the group consisting of captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, randolapril, and pharmaceutically acceptable salts of such compounds. In some embodiments, the ARBs are selected from the group consisting of losartan, candesartan, valsartan, eprosartan, telmisartan, and irbesartan.

In some embodiments, when an additional therapeutic agent is administered to a subject in combination with the peptide or mixture of peptides and PMO(s) and/or PPMO(s), a synergistic therapeutic effect is produced. For example, administration of the peptide or mixture of peptides and PMO(s) and/or PPMO(s) with one or more additional therapeutic agents for addressing the signs, symptoms, or severity of muscular dystrophy (e.g. DMD or BMD) will have greater than additive effects in the treatment of the disease. For example, lower doses of one or more of any individual therapeutic agent may be used in treating or preventing DMD or BMD, resulting in increased therapeutic efficacy and decreased side-effects. Alternatively, for example, higher doses of one or more of: (i) corticosteroids, (ii) ACE inhibitors, (iii) ARB(s), (iv) beta-blockers, (v) diuretics, and/or (vi) idebenone may be used than is otherwise tolerable because treatment with the peptide or mixture of peptides and PMO(s) and/or PPMO(s) described herein protects the subject from detrimental effects that otherwise affect the subject. In some embodiments, the synergistic effect will be improved ambulation (or delay in reduction in ambulation) resulting from the combined effects of increases in muscular dystrophin with increases in muscle function and energy associated with improved mitochondrial health of the subject (and the subject's muscles). In some embodiments, the synergistic effect will be extended life expectancy resulting from the combined effects of increases in muscular dystrophin resulting in improved muscle function (e.g., cardiac function) and energy associated with improved mitochondrial health of the subject (and the subject's muscles).

In any case, the therapeutic agents (e.g., a peptide (e.g., Compound A-1 or A-2) or mixture of peptides (e.g., Compound A-1 and A-2) in combination with PMO(s), e.g., Exondys 51® (Eteplirsen), Golodirsen (Vyondys 53™), Viltolarsen (Viltepso®), or Casimersen (Amondys 45™), or PPMO(s), may be administered in any order or even simultaneously. If simultaneously, the therapeutic agents may be provided in a single, unified form, or in multiple forms (by way of example only, as an IV injection or as two separate IV injections, or as a subcutaneous injection (e.g., for the peptide) and as an IV injection for the PMO(s) or PPMO(s)). One of the therapeutic agents may be given in multiple doses, or both may be given as multiple doses. If not simultaneous, the timing between the multiple doses may vary from more than zero weeks to less than four weeks. In addition, the combination methods, compositions, and formulations are not to be limited to the use of only two agents.

### Kits

The present technology also provides kits for treating DMD or BMD. In some embodiments, the kits comprise at least one peptide of Formula A (e.g., Compound A-1, A-2, A-3, A-4, A-5, A-6, A-7, or A-8, or mixtures thereof) and any suitable PMO or PPMO, packaged in a suitable container and optionally comprising instructions for its use.

It will be understood by one of ordinary skill in the relevant arts that other suitable modifications and adaptations to the compositions and methods described herein are readily apparent from the description of the technology contained herein in view of information known to the ordinarily skilled artisan, and may be made without departing from the scope of the present technology or any embodiment thereof. Having now described the present technology in detail, the same will be more clearly understood by reference to the following examples, which are included herewith for purposes of illustration only and are not intended to be limiting of the present technology.

### EXAMPLES

The present technology is further illustrated by the following examples, which should not be construed as limiting in any way. For each of the examples below, any peptide of Formula A (e.g., Compound A-1, A-2, A-3, A-4, A-5, A-6, A-7, or A-8, or mixtures thereof) or any suitable PMO or PPMO could be used. By way of example, but not by limitation, the aromatic-cationic peptide used in the example below could be H-D-Arg-2′6′-Dmt-Lys-Phe-NH₂ (SEQ ID NO: 2).

### Example 1—Use of H-D-Arg-2′6′-Dmt-Lys-Phe-NH₂ (SEQ ID NO: 2) in Combination with a Phosphorodiamidate Morpholino Oligomer (PMO) Augments Dystrophin Expression in a Mouse Model of Duchenne Muscular Dystrophy (DMD)

This Example demonstrates that the use of H-D-Arg-2′6′-Dmt-Lys-Phe-NH₂ (SEQ ID NO: 2) (MTP-131) in combination with a PMO is useful in the treatment of DMD.

### Study Design

Treatment groups. Four-week-old C57BL/10ScSnJ (stock #000476) and C57BL/10ScSn-Dmdmdx/J mice (stock #001801) were sourced from Jackson Laboratory in Bar Harbor, USA and sorted into a total of five treatment groups (Table 1). The C57BL/10ScSnJ (BL10) mice received vehicle #1 (1×PBS) and vehicle #2 (0.9% Saline). Of the C57BL/10ScSn-Dmdmdx/J (mdx) groups, one received vehicle #1(1×PBS) and vehicle #2 (0.9% Saline), the second group was treated with PMO (125 mg/kg) and vehicle #2 (0.9% Saline), the third group was treated with vehicle #1 (1×PSB) and MTP-131 (5 mg/kg), and the final group was treated with both PMO (125 mg/kg) and MTP-131 (5 mg/kg). PMO compound and the respective vehicle (1×PBS) was administered weekly via retro-orbital injection. MTP-131 compound and the vehicle (0.9% saline) were administered daily via intraperitoneal (IP) injection. Treatment was for a total of 7 weeks.

TABLE 1
Summary of treatment groups.

| | | | | N per |
|---|---|---|---|---|
| Group | Strain | Treatment | Dosing Route | group |
| 1 | BL10 | 1xPBS + | Retro-orbital injection | 15 |
| | | 0.9% Saline | (weekly) + IP injection | |
| | | | (daily) | |
| 2 | mdx | 1xPBS + | Retro-orbital injection | 15 |
| | | 0.9% Saline | (weekly) + IP injection | |
| | | | (daily) | |
| 3 | mdx | PMO (125 mg/kg) + | Retro-orbital injection | 15 |
| | | 0.9% Saline | (weekly) + IP injection | |
| | | | (daily) | |
| 4 | mdx | 1xPBS + | Retro-orbital injection | 15 |
| | | MTP-131 (5 mg/kg) | (weekly) + IP injection | |
| | | | (daily) | |
| 5 | mdx | PMO (125 mg/kg) + | Retro-orbital injection | 15 |
| | | MTP-131 (5 mg/kg) | (weekly) + IP injection | |
| | | | (daily) | |

Experimental endpoint measures. Upon arrival from Jackson laboratory to the Life Science Research Institute (LSRI) animal facility in Halifax, Canada, 4 week-old BL10 and mdx mice were subjected to a one-week acclimation period to the new facility, after which they were weighed, ear-tagged, and randomized. Once randomized, the animals were treated daily for 7 weeks. During treatment, mice were visually assessed for health (altered food and water consumption, abnormal breathing, circling, eye/hair matting and any other features of abnormal appearance). All treatment groups followed the experimental design outlined in Table 2. In vitro force measurements and takedowns were performed throughout weeks 6-7. Takedowns were staggered to accommodate the in vitro force schedule. Scheduling was representative of the treatment groups.

TABLE 2
Experimental design.

| | Weeks of Treatment | | | | | | |
|---|---|---|---|---|---|---|---|
| | (Weeks of Age) | | | | | | |
| Study | 1 (5) | 2 (6) | 3 (7) | 4 (8) | 5 (9) | 6 (10) | 7 (11) |
| Randomization | + | − | − | − | − | − | − |
| Body weights | + | + | + | + | + | + | + |
| Drug | + | + | + | + | + | + | + |
| administration | | | | | | | |
| In vitro | − | − | − | − | − | + | + |
| Blood/tissue | − | − | − | − | − | + | + |
| collection | | | | | | | |

### Materials and Methods

Animals. C57BL/10ScSnJ (stock #000476) and C57BL/10ScSn-Dmdmdx/J mice (stock #001801) were purchased from Jackson Laboratory in Bar Harbor, USA. The animals were acclimatized upon arrival to the animal facility for 7 days, housed in cages with up to 5 mice per cage.

Animal welfare. The animal work specified in this protocol was approved by the Dalhousie University Animal Committee in accordance with the Canadian Council on Animal Care.

During the study, 13-hour light/11-hour dark cycles were maintained. All testing was performed during the light cycle phase. The room temperature was maintained between 2° and 23° C. Food and water were available ad libitum for the duration of the study. Mice were identified by ear tags.

Randomization, blinding, and treatment groups. Randomization was done based on the average body weight of the cage. The mice were randomized into cages so that each cage had a similar average body weight (<5% difference in average body weight between cages). The treatment groups were assigned to these cages randomly.

Before performing any functional assessment or histological imaging and analysis, mice IDs were blinded by an independent associate who did not perform the assays. The samples were unblended only after the analysis was completed, prior to plotting of the data. An assortment of treatment groups are represented in all the blinded runs.

Treatment preparation—PMO. The phosphorodiamidate morpholino oligomer (PMO) was sourced from Gene Tools, LLC. The PMO used was mExon 23(+07-18) (5′-GGCCAAACCTCGGCTTACCTGAAAT-3′) (SEQ ID NO: 1) against the boundary sequences of exon and intron 23 of the mouse dystrophin gene. The PMO was prepared fresh weekly by diluting the compound in 1×PBS to the stock concentration of 100 mg/ml. Any remaining compound was stored at 4° C.

Treatment preparation—MTP-131. MTP-131 dosing formulation was prepared weekly and filtered through a 0.2 m polyether sulfone (PES) filter. Because MTP-131 is extremely hygroscopic it was conditioned before use and weighed immediately after. If possible, MTP-131 was weighed in a low moisture environment (<20% RH). Prior to use, MTP-131 was removed from the refrigerator. The un-opened bottle was placed in a low moisture environment (<20% RH) and allowed to equilibrate at room temperature for at least 2 hours. Quickly, an aliquot was weighed for use without returning any un-used standard to the bottle and the bottle was immediately tightly capped. Immediately after use, the inside of the bag was wiped with a paper towel to remove any moisture, and the reference bottle (tightly capped) was placed back into the aluminum bag, re-sealed and placed back in 2-8° C. storage.

Treatment administration—PMO. PMO compound was delivered weekly via retro-orbital intravenous injection using a 28-gauge needle. Mice were gently removed from their cage and anesthetized using 5% isoflurane and maintained with 1.5-2% isoflurane. Once the pedal reflex was absent, the mouse was positioned on its side, and loose skin behind the shoulders and ears pinned back using the thumb and middle finger of the non-dominant hand. The skin surrounding the eye was drawn back using the index finger, allowing the eye to protrude slightly. The needle was inserted carefully at a 30° angle with the bevel away from the eye, lateral to the medial canthus, through the conjunctival membrane. Once in the retro-orbital sinus, the compound was slowly injected (10 μl/5-10 seconds). The needle was then carefully removed to prevent injury and leakage. If more than 20 μl of leakage occurred, the injectable was aspirated back into the syringe and injected again. Once the needle was removed, the eyelid was closed, and mild pressure applied to the injection site. After dosing, the animals were returned to their cage and monitored until the effects of anesthesia wore off and monitored for any injection-related complications, including lethargy, swelling, visible trauma or seizing.

Treatment administration—MTP-131. MTP-131 was delivered via intraperitoneal (IP) injection, which was performed by restraining the mouse and administering the treatment on either side of the abdomen, halfway between the midline and natural bend of a knee. The needle was inserted at a 450 angle, bevel side up. The syringe was aspirated slightly before the treatment was delivered to ensure a proper intraperitoneal position. Once the injection was delivered, the mouse was monitored for a couple of minutes for any adverse side effects. Animals received an injection on alternating sides daily to reduce damage from repeat injections.

Body weights. Body weights were measured weekly, starting after acclimation of the animals to the facility. At the start of each body weight collection, an OHAUS Scout® Pro digital scale was tared to an open 750 mL Tupperware container. Mice were constrained individually to the container and placed on top of the OHAUS Scout® Pro scale to measure the body weight in grams.

In vitro force measurements. Contractile properties were measured in vitro on the right extensor digitorum longus (EDL) muscle at the end of the study (weeks 6-7). The mice were anesthetized using ketamine and xylazine. The EDL muscle of the right hindlimb was removed from each mouse and immersed in individual oxygenated baths (95% 02, 5% CO₂) that contain Ringer's solution (pH 7.4) at 25° C. The muscle was flanked by two electrodes. Using non-fatiguing twitches, the muscle was maintained at 10 mN for force generation. The maximal force was measured with the muscle held at 10 mN. The muscle was stimulated with an electrode to elicit tetanic contractions that are separated by 2 minute rest intervals. For the EDL, with each subsequent tetanus, the stimulation frequency was increased in steps of 20, 30 or 50 Hz until the force reached a plateau (usually occurring around 250 Hz). That plateau was considered the maximum force (mN) generated by the muscle. The muscle's cross-sectional area was measured based on muscle mass, muscle length, and tissue density. Finally, the muscle specific force (kN/m2) was calculated based on the cross-sectional area of the muscle.

Terminal blood collection. Terminal blood collection was performed via cardiac puncture. The level of sedation induced by ketamine/xylazine was checked prior to cardiac puncture using a pedal reflex test. If the mouse reacted to the pedal reflex test, more anesthesia was injected, and another pedal reflex test was done. Once the mouse was unresponsive to the pedal reflex test, the cardiac puncture was performed using a 1 mL, 27.5-gauge syringe. The blood was divided into two tubes: ½ into a K2EDTA tube for plasma and ½ into a 1.5 mL Eppendorf tube for serum, and immediately placed on ice. At the end of the dissection, blood samples were centrifuged at 10,000 RPM for 10 minutes at 4° C., and serum and plasma was be stored at −80° C.

Tissue collection. After the blood was collected, the mice were euthanized via cervical dislocation. The tibialis anterior (TA), quadriceps, gastrocnemius, soleus, and extensor digitorum longus (EDL) were collected bilaterally. The heart and diaphragm were also collected. Tissues were weighed once collected, except for the diaphragm. The tissues were either mounted on a cue card or stored in foil and frozen in liquid nitrogen-cooled isopentane as described in Table 3. All frozen tissues were immediately placed on dry ice, then stored in 15 ml falcon tubes, and transferred at the completion of dissection to a −80° C. freezer.

TABLE 3
Tissue processing summary.

| | Isopentane-Frozen | |
|---|---|---|
| | (mounted on | Isopentane-Frozen |
| Tissue | cue card) | (stored in foil) |
| Heart | ½ (base) | ½ (apex) |
| Diaphragm | ½ | ½ |
| TA (right and left) | ½ | ½ |
| Quadriceps (right and left) | ½ | ½ |
| Gastrocnemius (right and left) | ½ | ½ |
| Soleus (right and left) | ½ | ½ |
| EDL (right) | N/A | Full |
| EDL (left) | ½ | ½ |

Western blot. TA muscle sections were lysed in an extraction buffer (10% sodium dodecyl sulfate, 0.1 M tris buffer, 15 mM dithiothreitol), and protein quantitation was performed using the BCA Protein Assay Kit (Pierce). To extract protein, the muscle was cryosectioned at 10 μm thick, with at least 10 sections per sample. Sections were lysed using the extraction buffer and vortexed for 40 seconds, heated at 90° C. for 2 minutes, followed by centrifugation at 14,000 rpm for 10 minutes at room temperature. Once the protein concentration was determined using the BCA kit, each sample was loaded at 25 μg of protein per well. The standard curve was generated using a mix of five wild type (BL10) samples, serially diluted with mdx samples to obtain 20%, 10%, 5%, 2.5%, 1% and 0% dystrophin. The gels were run using NuPAGE 3-8% Tris-Acetate Midi gels and tris-acetate running buffer for 1.25 hours at 150 V. The transfer of the protein to nitrocellulose membrane was performed using wet transfer for 4 hours at 0.3 Amps with constant stirring. After the transfer, the membrane was dried overnight, and the gel was stained with Coomassie Blue dye to obtain the housekeeping normalization protein, myosin heavy chain. The membrane was blocked in 5% skim milk for 1 hour at room temperature and probed with rabbit anti-dystrophin antibody (Abcam; Cat #ab15277) for 3 hours with constant shaking. The membrane was then be washed 3 times PBST for 10 minutes at room temperature and re-blocked with 5% skim milk for 5 minutes on the shaker. The anti-rabbit secondary antibody was added to the blots for 1 hour. ECL detection reagents were added, and images of the bands were taken using the ChemiDoc system (Biorad). The gels were analyzed for percent dystrophin relative to normal. The results were presented as percent dystrophin for each sample (mouse) ID. Summaries of the Western blot runs are provided in Tables 4-6.

TABLE 4
Summary of Western blot Run 1.

| Mouse ID | Strain | Treatment Group | Gel # | Run No |
|---|---|---|---|---|
| 2781 | Mdx | 1xPBS + 0.9% Saline | 5 | 1 |
| 2749 | Mdx | 1xPBS + 0.9% Saline | | |
| 2735 | Mdx | PMO (125 mg/kg) + 0.9% Saline | | |
| 2790 | Mdx | PMO (125 mg/kg) + 0.9% Saline | | |
| 2719 | Mdx | 1xPBS + MTP-131 (5 mg/kg) | | |
| 2780 | Mdx | 1xPBS + MTP-131 (5 mg/kg) | | |
| 2712 | Mdx | 1xPBS + MTP-131 (5 mg/kg) | | |
| 2732 | Mdx | PMO (125 mg/kg) + MTP-131 | | |
| | | (5 mg/kg) | | |
| 2721 | Mdx | PMO (125 mg/kg) + MTP-131 | | |
| | | (5 mg/kg) | | |
| 2800 | Mdx | PMO (125 mg/kg) + MTP-131 | | |
| | | (5 mg/kg) | | |
| 2703 | Mdx | 1xPBS + 0.9% Saline | 6 | |
| 2710 | Mdx | 1xPBS + 0.9% Saline | | |
| 2782 | Mdx | 1xPBS + 0.9% Saline | | |
| 2739 | Mdx | PMO (125 mg/kg) + 0.9% Saline | | |
| 2702 | Mdx | PMO (125 mg/kg) + 0.9% Saline | | |
| 2765 | Mdx | 1xPBS + MTP-131 (5 mg/kg) | | |
| 2744 | Mdx | 1xPBS + MTP-131 (5 mg/kg) | | |

TABLE 5
Summary of Western blot Run 2.*

| Mouse ID | Strain | Treatment Group | Gel # | Run No |
|---|---|---|---|---|
| 2747 | Mdx | 1xPBS + 0.9% Saline | 7 | 2 |
| 2787 | Mdx | 1xPBS + 0.9% Saline | | |
| 2726 | Mdx | PMO (125 mg/kg) + 0.9% Saline | | |
| 2772 | Mdx | PMO (125 mg/kg) + 0.9% Saline | | |
| 2779 | Mdx | PMO (125 mg/kg) + 0.9% Saline | | |
| 2755 | Mdx | 1xPBS + MTP-131 (5 mg/kg) | | |
| 2773 | Mdx | 1xPBS + MTP-131 (5 mg/kg) | | |
| 2734 | Mdx | PMO (125 mg/kg) + MTP-131 | | |
| | | (5 mg/kg) | | |
| 2733 | Mdx | PMO (125 mg/kg) + MTP-131 | | |
| | | (5 mg/kg) | | |
| 2706 | Mdx | 1xPBS + 0.9% Saline | 8 | |
| 2791 | Mdx | 1xPBS + 0.9% Saline | | |
| 2795 | Mdx | 1xPBS + 0.9% Saline | | |
| 2761 | Mdx | PMO (125 mg/kg) + 0.9% Saline | | |
| 2715 | Mdx | PMO (125 mg/kg) + 0.9% Saline | | |
| 2757 | Mdx | 1xPBS + MTP-131 (5 mg/kg) | | |
| 2759 | Mdx | 1xPBS + MTP-131 (5 mg/kg) | | |
| 2736 | Mdx | PMO (125 mg/kg) + MTP-131 | | |
| | | (5 mg/kg) | | |
| 2720 | Mdx | PMO (125 mg/kg) + MTP-131 | | |
| | | (5 mg/kg) | | |
| 2758 | Mdx | PMO (125 mg/kg) + MTP-131 | | |
| | | (5 mg/kg) | | |

*Western blot reruns were required for Run 2 and 3. Gels are numbered 5-10.

TABLE 6
Summary of Western blot Run 3.*

| Mouse ID | Strain | Treatment Group | Gel # | Run No |
|---|---|---|---|---|
| 2753 | Mdx | 1xPBS + 0.9% Saline | 9 | 3 |
| 2794 | Mdx | 1xPBS + 0.9% Saline | | |
| 2738 | Mdx | PMO (125 mg/kg) + 0.9% Saline | | |
| 2743 | Mdx | PMO (125 mg/kg) + 0.9% Saline | | |
| 2769 | Mdx | 1xPBS + MTP-131 (5 mg/kg) | | |
| 2768 | Mdx | 1xPBS + MTP-131 (5 mg/kg) | | |
| 2760 | Mdx | 1xPBS + MTP-131 (5 mg/kg) | | |
| 2762 | Mdx | PMO (125 mg/kg) + MTP-131 | | |
| | | (5 mg/kg) | | |
| 2737 | Mdx | PMO (125 mg/kg) + MTP-131 | | |
| | | (5 mg/kg) | | |
| 2756 | Mdx | PMO (125 mg/kg) + MTP-131 | | |
| | | (5 mg/kg) | | |
| 2777 | Mdx | 1xPBS + 0.9% Saline | 10 | |
| 2792 | Mdx | 1xPBS + 0.9% Saline | | |
| 2784 | Mdx | PMO (125 mg/kg) + 0.9% Saline | | |
| 2745 | Mdx | PMO (125 mg/kg) + 0.9% Saline | | |
| 2741 | Mdx | PMO (125 mg/kg) + 0.9% Saline | | |
| 2751 | Mdx | 1xPBS + MTP-131 (5 mg/kg) | | |
| 2793 | Mdx | 1xPBS + MTP-131 (5 mg/kg) | | |
| 2714 | Mdx | PMO (125 mg/kg) + MTP-131 | | |
| | | (5 mg/kg) | | |
| 2713 | Mdx | PMO (125 mg/kg) + MTP-131 | | |
| | | (5 mg/kg) | | |
| 2729 | Mdx | PMO (125 mg/kg) + MTP-131 | | |
| | | (5 mg/kg) | | |
| 2740 | Mdx | PMO (125 mg/kg) + MTP-131 | | |
| | | (5 mg/kg) | | |

*Western blot reruns were required for Run 2 & 3. Gels are numbered 5-10.

Histological analysis—Immunofluorescence—Dystrophin. TA muscle (n=8/group) was sectioned at 10 μm (2 sections/slide) and stored at −80° C. until the staining was performed. Slides were stained with an anti-dystrophin antibody. Representative images were provided for each treatment group.

Histological analysis—H&E—Inflammation Analysis. TA muscle (n=8/group) was sectioned at 10 μm (2 sections/slide) and stored at −80° C. until the staining was performed. Slides were stained with Hematoxylin & Eosin (H&E) and were analyzed for percent inflammation. An inflammatory focus (pl. foci) was measured by counting 10 or more inflammatory cells in a single cluster. These foci areas were measured in mm2 and then compared to the tissue's total area, from which a percentage was calculated. This percentage was interpreted as the percent inflammation of the tissue (%).

Statistical analysis. The nominal level for defining statistical significance is p<0.05. Normality was tested for each outcome within each treatment group using both the Shapiro-Wilk normality test and a visual inspection of each histogram. Comparisons between BL10 vehicle and mdx vehicle were performed using two-sample student's t-tests for equal variances. Comparisons between mdx treatment groups and the mdx vehicle group were performed using individual t-tests but with an adjustment of resulting p-values for multiple comparisons (Sidak method). This analysis is analogous to an ANOVA with a Dunnet's post-hoc test to account for all groups compared to a control. Analysis of body weights were performed for each week and over time. Analysis of body weights used longitudinal linear regression models which account for repeated measurements within each mouse.

### Results

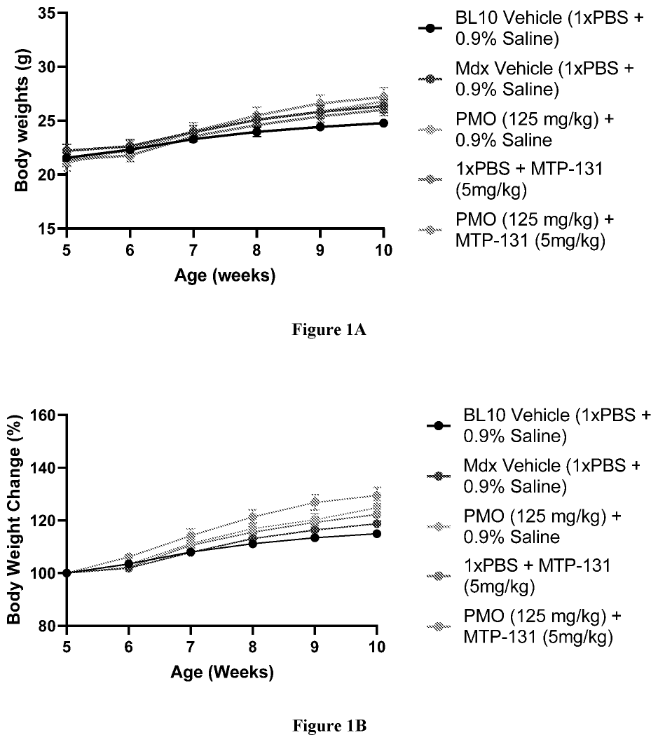

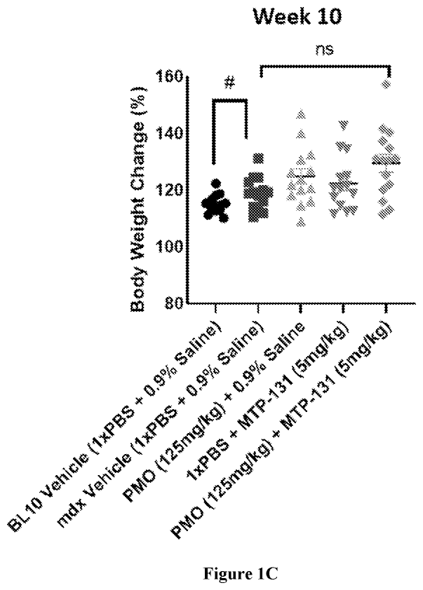

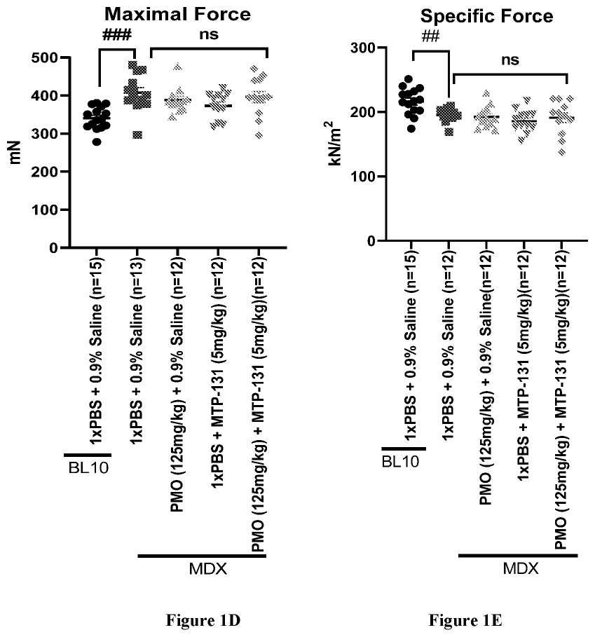

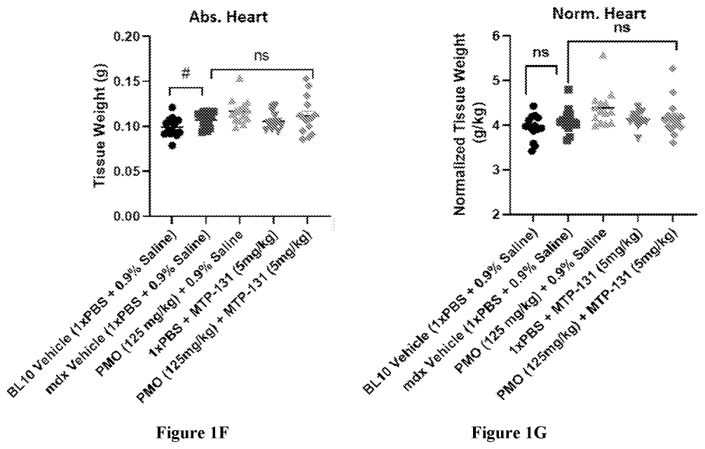

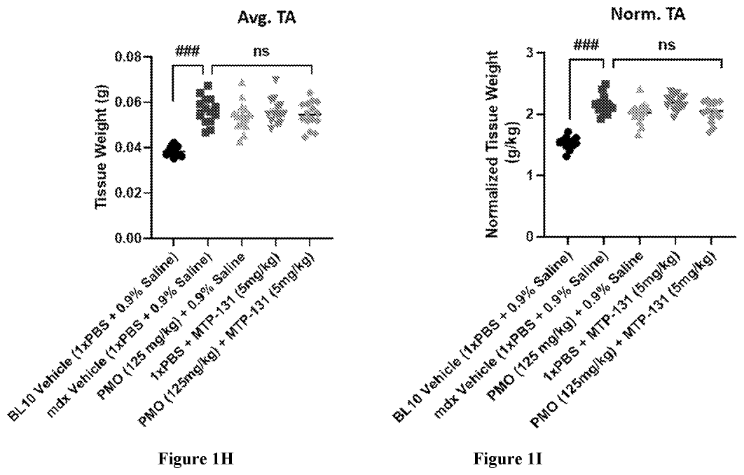

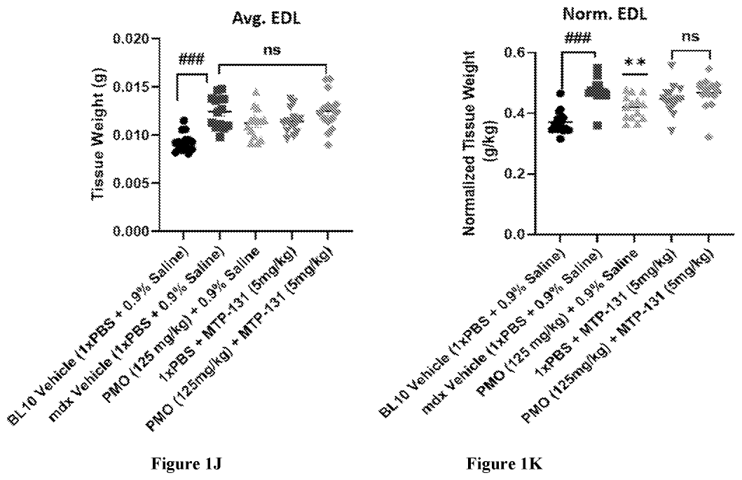

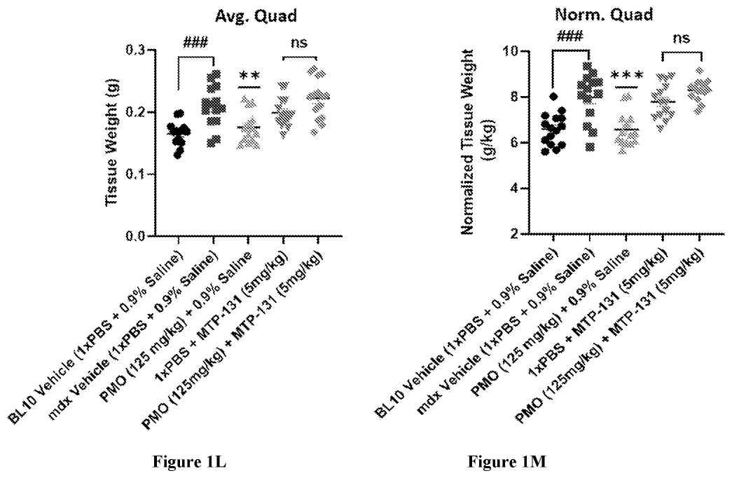

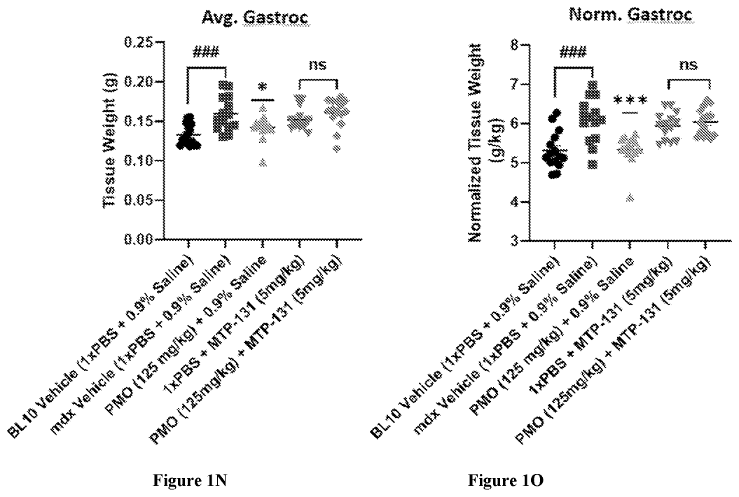

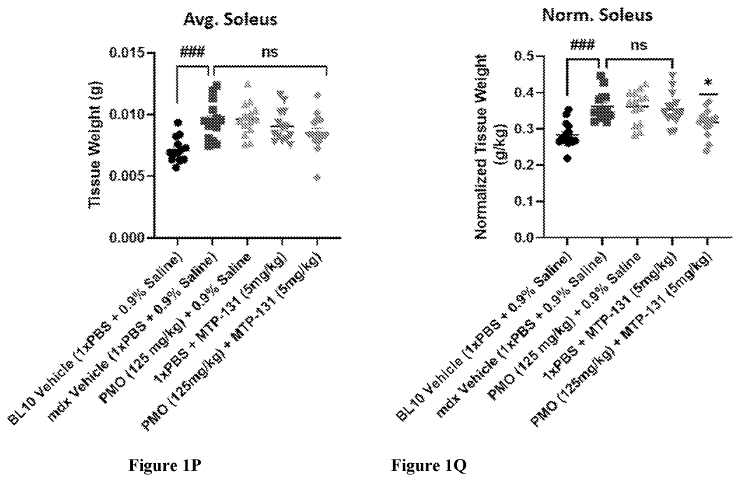

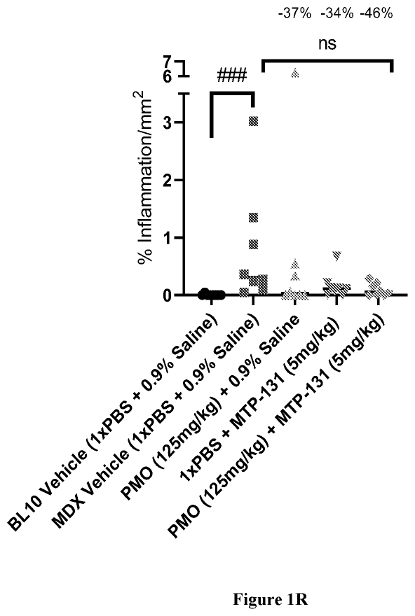

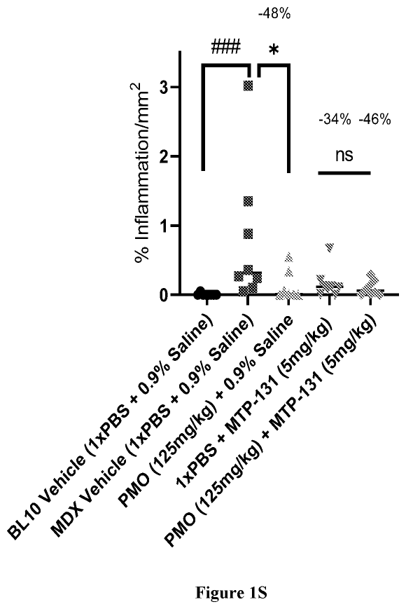

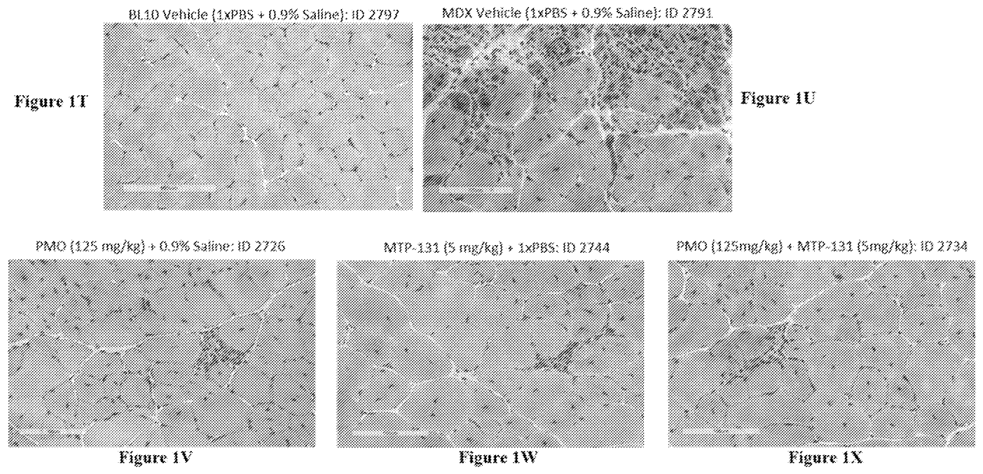

Body weights. The body weights and percent body weight change per week per treatment group are provided in FIGS. 1A and 1B, respectively. The percent body weight change at week 10 for each treatment group is provided in FIG. 1C. As shown in FIGS. 1A-1B, increases in body weight were observed in all mdx mice regardless of treatment.

In vitro force measurements. In vitro measurements of the maximal force (Nm) and specific force (kN/m²) generated in the right extensor digitorum longus (EDL) are shown in FIGS. 1D and 1E, respectively.

Tissue weights. The average tissue weight normalized to bodyweight per treatment group (g/kg) for the heart, tibialis anterior (TA), extensor digitorum longus (EDL), quadriceps (quad), gastrocnemius (gastroc), and soleus are shown in FIGS. 1F-1Q.

Tissue inflammation. As shown in FIGS. 1R-1X, inflammation was reduced in the tibialis anterior muscles from each of the following groups: (PMO (125 mg/kg)+0.9% Saline); (1×PBS+MTP-131 (5 mg/kg)); and (PMO (125 mg/kg)+MTP-131 (5 mg/kg)), as compared to the mdx vehicle group (1×PBS+0.9% Saline).

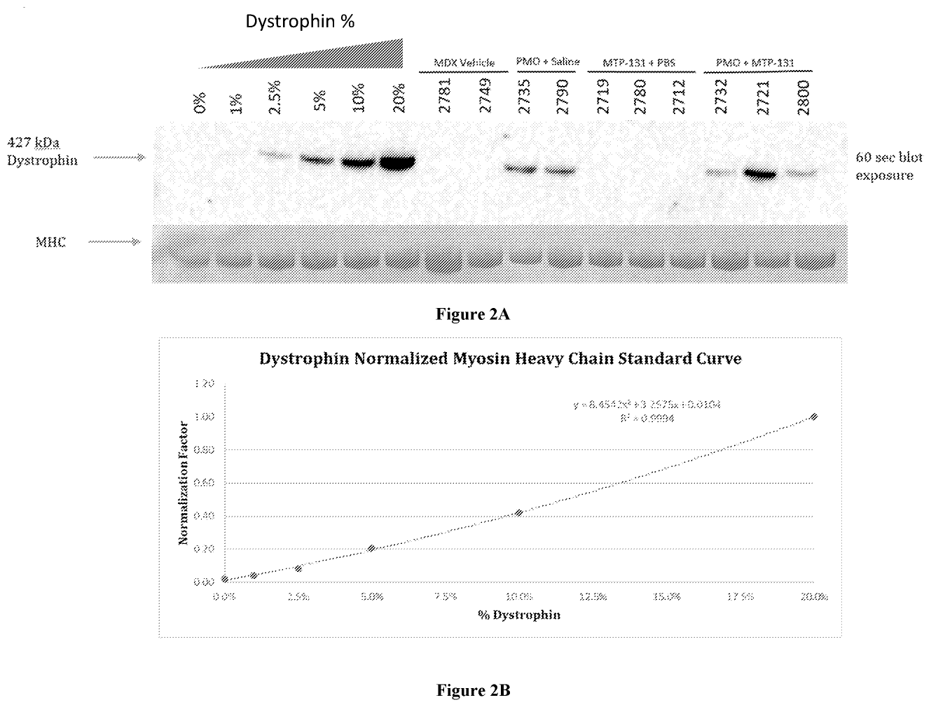

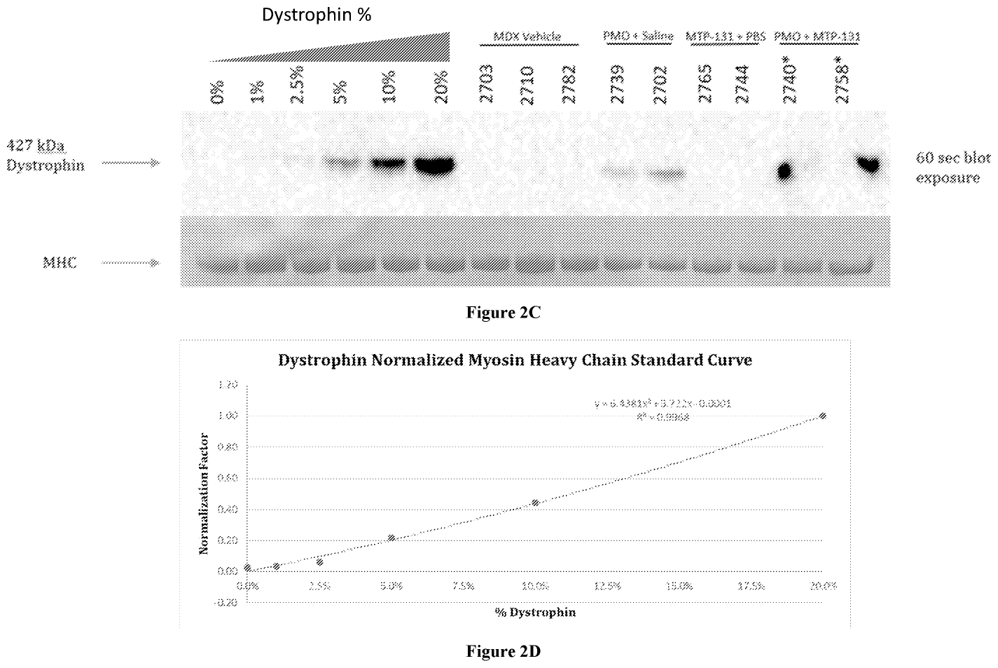

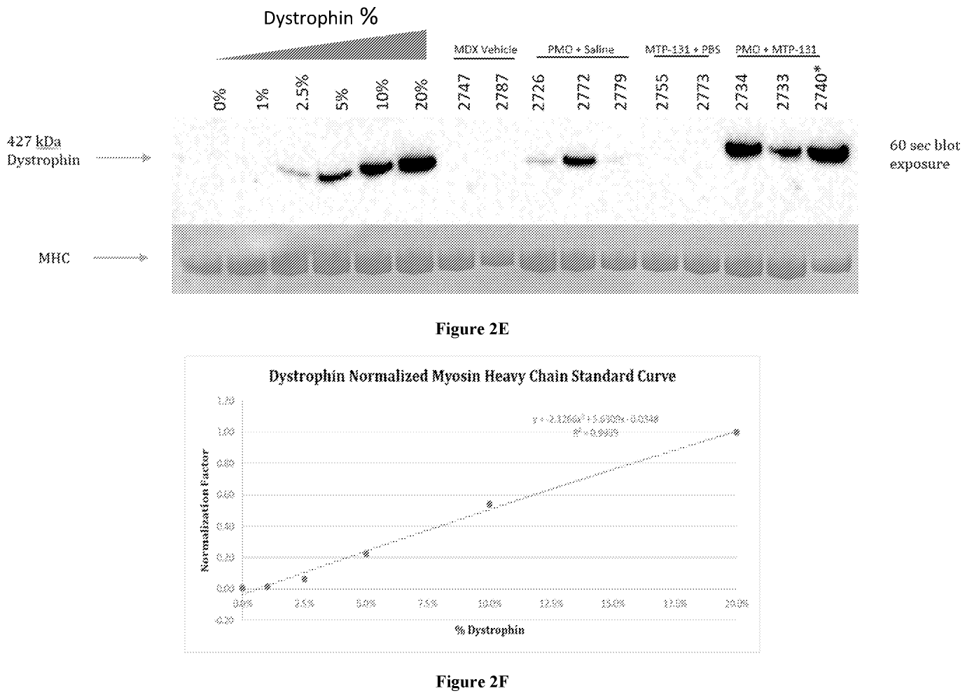

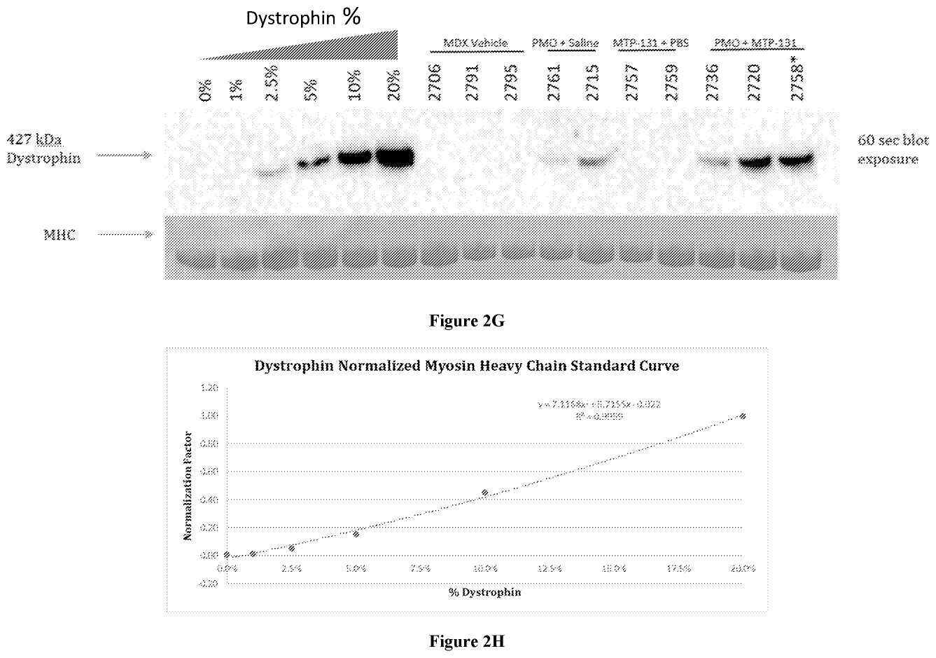

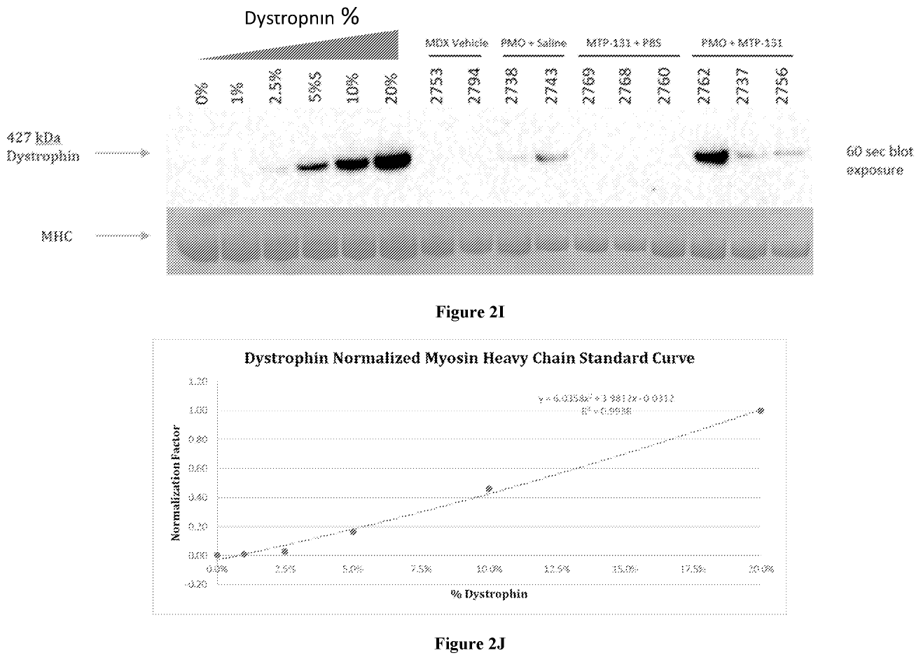

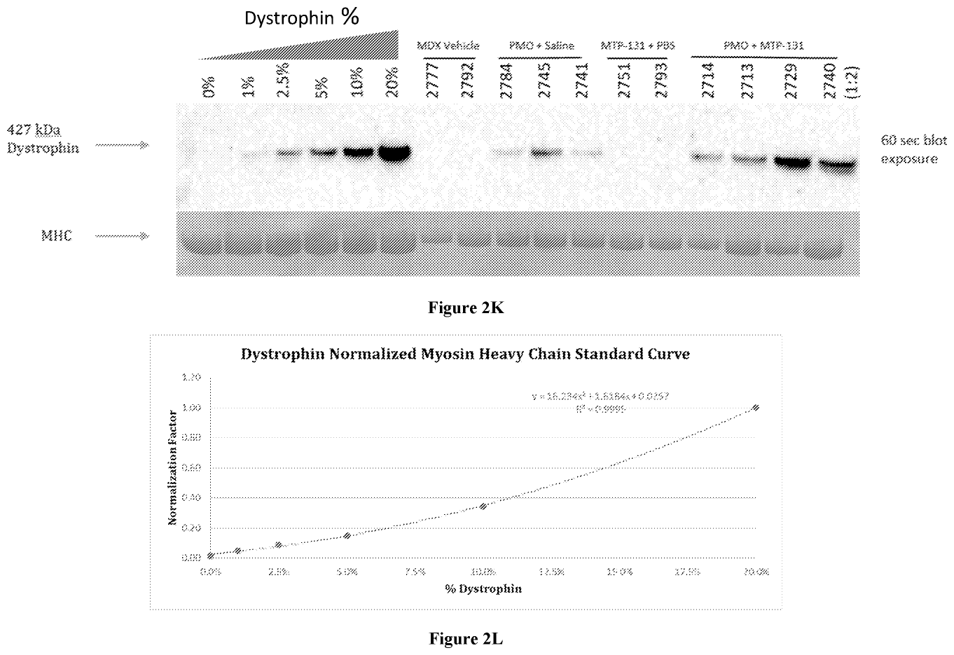

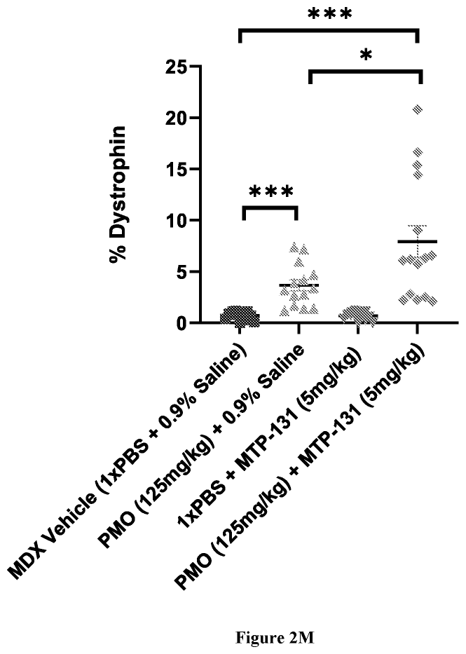

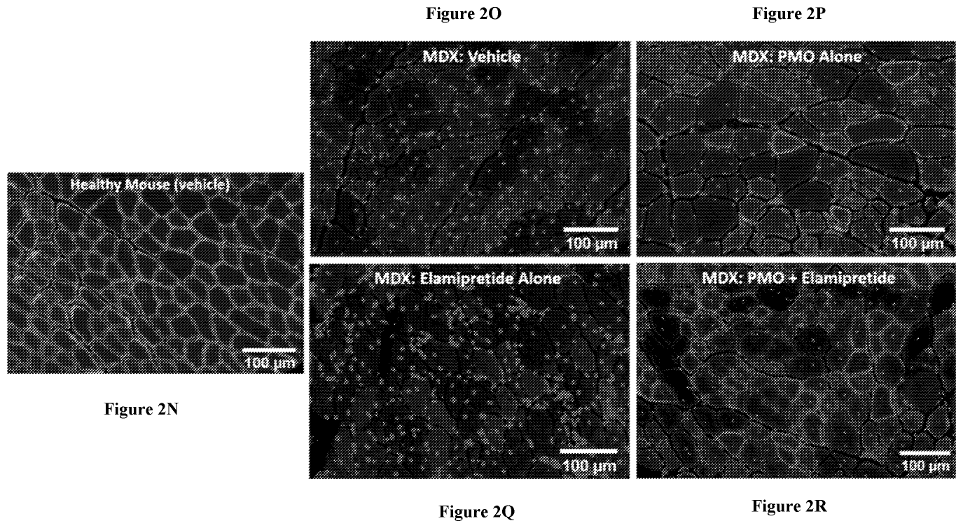

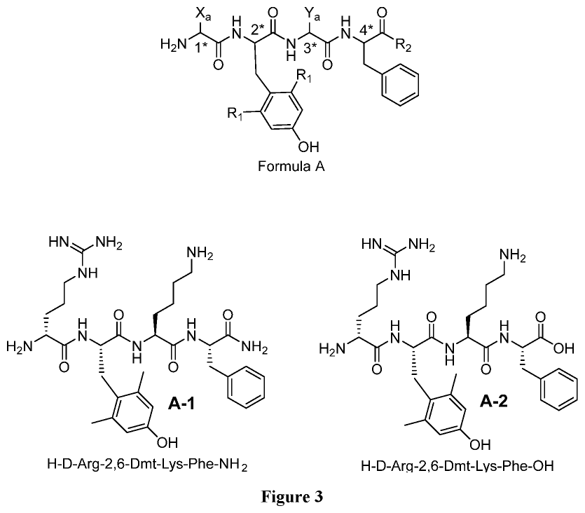

Western blots. The quantitative data for the Western blots performed according to the summaries provided in Tables 4-6 above are shown in Tables 7-12 below, which correspond to FIGS. 2A, 2C, 2E, 2G, 2I, and 2K, respectively.

TABLE 7
Western Blot Results - Run 1, Gel #5 -
Tibialis Anterior (see FIG. 2A)

| Treatment Group | Mouse ID | % Dystrophin |
|---|---|---|
| 1xPBS + 0.9% Saline | 2781 | 0.0% |
| 1xPBS + 0.9% Saline | 2749 | 0.3% |
| PMO (125 mg/kg) + 0.9% Saline | 2735 | 6.0% |
| PMO (125 mg/kg) + 0.9% Saline | 2790 | 4.2% |
| 1xPBS + MTP-131 (5 mg/kg) | 2719 | 0.3% |
| 1xPBS + MTP-131 (5 mg/kg) | 2780 | 0.4% |
| 1xPBS + MTP-131 (5 mg/kg) | 2712 | 0.6% |
| PMO (125 mg/kg) + MTP-131 (5 mg/kg) | 2732 | 2.1% |
| PMO (125 mg/kg) + MTP-131 (5 mg/kg) | 2721 | 9.0% |
| PMO (125 mg/kg) + MTP-131 (5 mg/kg) | 2800 | 2.2% |

TABLE 8
Western Blot Results - Run 1, Gel #6 -
Tibialis Anterior (see FIG. 2C*)

| Treatment Group | Mouse ID | % Dystrophin |
|---|---|---|
| 1xPBS + 0.9% Saline | 2703 | 1.2% |
| 1xPBS + 0.9% Saline | 2710 | 1.0% |
| 1xPBS + 0.9% Saline | 2782 | 1.0% |
| PMO (125 mg/kg) + 0.9% Saline | 2739 | 2.7% |
| PMO (125 mg/kg) + 0.9% Saline | 2702 | 4.7% |
| 1xPBS + MTP-131 (5 mg/kg) | 2765 | 1.0% |
| 1xPBS + MTP-131 (5 mg/kg) | 2744 | 1.2% |

*Samples 2740 and 2758 shown in FIG. 2C were repeated due to a bubble in the blot. 2758 was repeated in Run 2, Gel (or blot) #8. 2740 was repeated in Run 2, Gel (or blot) #7 (undiluted) and was then run again in Run 3, Gel (or blot) #10 (diluted 1:2).

TABLE 9
Western Blot Results - Run 2, Gel #7 -
Tibialis Anterior (see FIG. 2E)

| Treatment Group | Mouse ID | % Dystrophin |
|---|---|---|
| 1xPBS + 0.9% Saline | 2747 | 0.8% |
| 1xPBS + 0.9% Saline | 2787 | 0.8% |
| PMO (125 mg/kg) + 0.9% Saline | 2726 | 1.7% |
| PMO (125 mg/kg) + 0.9% Saline | 2772 | 7.4% |
| PMO (125 mg/kg) + 0.9% Saline | 2779 | 1.2% |
| 1xPBS + MTP-131 (5 mg/kg) | 2755 | 0.8% |
| 1xPBS + MTP-131 (5 mg/kg) | 2773 | 0.8% |
| PMO (125 mg/kg) + MTP-131 (5 mg/kg) | 2734 | 16.6% |
| PMO (125 mg/kg) + MTP-131 (5 mg/kg) | 2733 | 5.7% |
| PMO (125 mg/kg) + MTP-131 (5 mg/kg) | 2740 | 28.0%* |

*2740 diluted 1:2 in Run 3 Gel (or blot) #10 as above result was outside the standard curve range.

TABLE 10
Western Blot Results - Run 2, Gel #8 -
Tibialis Anterior (see FIG. 2G)

| Treatment Group | Mouse ID | % Dystrophin |
|---|---|---|
| 1xPBS + 0.9% Saline | 2706 | 0.7% |
| 1xPBS + 0.9% Saline | 2791 | 0.8% |
| 1xPBS + 0.9% Saline | 2795 | 0.9% |
| PMO (125 mg/kg) + 0.9% Saline | 2761 | 1.4% |
| PMO (125 mg/kg) + 0.9% Saline | 2715 | 3.2% |
| 1xPBS + MTP-131 (5 mg/kg) | 2757 | 0.8% |
| 1xPBS + MTP-131 (5 mg/kg) | 2759 | 0.8% |
| PMO (125 mg/kg) + MTP-131 (5 mg/kg) | 2736 | 2.5% |
| PMO (125 mg/kg) + MTP-131 (5 mg/kg) | 2720 | 6.6% |
| PMO (125 mg/kg) + MTP-131 (5 mg/kg) | 2758 | 6.3%* |

*2758 was repeated in this run. Originally ran in Run 1 Gel (or blot) #6.

TABLE 11
Western Blot Results - Run 3, Gel #9 -
Tibialis Anterior (see FIG. 2I)

| Treatment Group | Mouse ID | % Dystrophin |
|---|---|---|
| 1xPBS + 0.9% Saline | 2753 | 0.9% |
| 1xPBS + 0.9% Saline | 2794 | 1.0% |
| PMO (125 mg/kg) + 0.9% Saline | 2738 | 1.4% |
| PMO (125 mg/kg) + 0.9% Saline | 2743 | 3.9% |
| 1xPBS + MTP-131 (5 mg/kg) | 2769 | 1.0% |
| 1xPBS + MTP-131 (5 mg/kg) | 2768 | 1.0% |
| 1xPBS + MTP-131 (5 mg/kg) | 2760 | 1.0% |
| PMO (125 mg/kg) + MTP-131 (5 mg/kg) | 2762 | 14.4% |
| PMO (125 mg/kg) + MTP-131 (5 mg/kg) | 2737 | 2.6% |
| PMO (125 mg/kg) + MTP-131 (5 mg/kg) | 2756 | 2.3% |

TABLE 12
Western Blot Results - Run 3, Gel #10 -
Tibialis Anterior (see FIG. 2K)

| Treatment Group | Mouse ID | % Dystrophin |
|---|---|---|
| 1xPBS + 0.9% Saline | 2777 | 0.0% |
| 1xPBS + 0.9% Saline | 2792 | 0.0% |
| PMO (125 mg/kg) + 0.9% Saline | 2784 | 2.9% |
| PMO (125 mg/kg) + 0.9% Saline | 2745 | 7.2% |
| PMO (125 mg/kg) + 0.9% Saline | 2741 | 3.1% |
| 1xPBS + MTP-131 (5 mg/kg) | 2751 | 0% |
| 1xPBS + MTP-131 (5 mg/kg) | 2793 | 0% |
| PMO (125 mg/kg) + MTP-131 (5 mg/kg) | 2714 | 6.2% |
| PMO (125 mg/kg) + MTP-131 (5 mg/kg) | 2713 | 6.1% |
| PMO (125 mg/kg) + MTP-131 (5 mg/kg) | 2729 | 15.4% |
| PMO (125 mg/kg) + MTP-131 (5 mg/kg) | 2740 (1:2) | 10.4% |
| | 2740 | 20.8% |

Table 13 provides a summary of the Western blot data shown in Tables 7-12, organized by treatment group. As shown in Table 13 and FIG. 2M, treatment with MTP-131 in combination with PMO resulted in a significant increase in dystrophin expression in DMD mice when compared to the administration of PMO alone.

TABLE 13
Western Blot % Dystrophin Summary - Tibialis Anterior

| | Mouse | % | | Std. |
|---|---|---|---|---|
| Treatment | ID | Dystrophin | Average | Dev. |
| MDX Vehicle (1xPBS + | 2753 | 1.10% | 0.73% | 0.38% |
| 0.9% Saline) | 2794 | 1.10% | | |
| | 2777 | 0.40% | | |
| | 2792 | 0.10% | | |
| | 2747 | 0.80% | | |
| | 2787 | 0.80% | | |
| | 2706 | 0.70% | | |
| | 2791 | 0.80% | | |
| | 2795 | 0.90% | | |
| | 2781 | 0.00% | | |
| | 2749 | 0.30% | | |
| | 2703 | 1.20% | | |
| | 2710 | 1.00% | | |
| | 2782 | 1.00% | | |
| PMO (125 mg/kg) + | 2738 | 1.40% | 3.66% | 2.07% |
| 0.9% Saline | 2743 | 3.90% | | |
| | 2784 | 2.80% | | |
| | 2745 | 7.20% | | |
| | 2741 | 3.40% | | |
| | 2726 | 1.70% | | |
| | 2772 | 7.40% | | |
| | 2779 | 1.20% | | |
| | 2761 | 1.40% | | |
| | 2715 | 3.20% | | |
| | 2735 | 6.00% | | |
| | 2790 | 4.20% | | |
| | 2739 | 2.70% | | |
| | 2702 | 4.70% | | |
| 1xPBS + MTP-131 | 2769 | 1.00% | 0.70% | 0.39% |
| (5 mg/kg) | 2768 | 1.00% | | |
| | 2760 | 1.10% | | |
| | 2751 | 0.00% | | |
| | 2793 | 0.00% | | |
| | 2755 | 0.80% | | |
| | 2773 | 0.80% | | |
| | 2757 | 0.80% | | |
| | 2759 | 0.80% | | |
| | 2719 | 0.30% | | |
| | 2780 | 0.40% | | |
| | 2712 | 0.60% | | |
| | 2765 | 1.00% | | |
| | 2744 | 1.20% | | |
| PMO (125 mg/kg) + | 2762 | 14.40% | 7.93% | 6.03% |
| MTP-131 (5 mg/kg) | 2737 | 2.80% | | |
| | 2756 | 2.30% | | |
| | 2714 | 6.20% | | |
| | 2713 | 6.10% | | |
| | 2729 | 15.40% | | |
| | 2740 | 20.80% | | |
| | 2734 | 16.60% | | |
| | 2733 | 5.70% | | |
| | 2736 | 2.50% | | |
| | 2720 | 6.60% | | |
| | 2758 | 6.30% | | |
| | 2732 | 2.10% | | |
| | 2721 | 9.00% | | |
| | 2800 | 2.20% | | |

In addition, as shown in FIGS. 2N-2R, treatment with MTP-131 in combination with PMO resulted in an increase in dystrophin+muscle fibers in tibialis anterior (TA) muscle sections in MDX mice, as shown by the encapsulation around the muscle fibers (see FIGS. 2N, 2P and 2Q).

Accordingly, these results demonstrate that MTP-131 in combination with an exon-skipping PMO is useful in methods for increasing dystrophin expression and treating muscular dystrophy, such as DMD or BMD.

### Example 2—Use of H-D-Arg-2′6′-Dmt-Lys-Phe-NH₂ (SEQ ID NO: 2) or H-D-Arg-2′6′-Dmt-Lys-Phe-OH (SEQ ID NO: 3) in Combination with a Phosphorodiamidate Morpholino Oligomer (PMO) for the Treatment of Muscular Dystrophy in Human Subjects

This example demonstrates the use of an effective amount of H-D-Arg-2′6′-Dmt-Lys-Phe-NH₂ (SEQ ID NO: 2) or H-D-Arg-2′6′-Dmt-Lys-Phe-OH (SEQ ID NO: 3) in combination with an effective amount of an appropriately designed exon-skipping PMO that is relevant to the dystrophin lesion in a subject in need thereof, wherein the subject has been diagnosed with or is suspected of having a muscular dystrophy, such as DMD or BMD. By way of example, but not by limitation, the PMO comprises an antisense oligomer of about 20-50 nucleotides in length, or a pharmaceutically acceptable salt thereof, capable of binding a selected target in human dystrophin pre-mRNA to induce exon skipping in the human dystrophin gene, wherein the antisense oligomer comprises a sequence of bases that specifically hybridizes to a dystrophin exon target region. By way of example, but not by limitation, the PMO may be chemically linked to a cell penetrating peptide that improves cellular uptake of the PMO (e.g., a PPMO). Illustrative, non-limiting examples of PMOs include any one or more the PMOs selected from Eteplirsen (Exondys 51®), Golodirsen (Vyondys 53™), Viltolarsen (Viltepso®), and Casimersen (Amondys 45™). Two or more antisense oligomers may be used together to induce exon skipping of single or multiple exons.

### Methods

Subjects suspected of having or diagnosed as having DMD or BMD receive daily subcutaneous administration of H-D-Arg-2′6′-Dmt-Lys-Phe-NH₂ (SEQ ID NO: 2) or H-D-Arg-2′6′-Dmt-Lys-Phe-OH (SEQ ID NO: 3) (e.g., 0.5-5.0 mg/kg/day) and weekly intravenous (IV) PMO (or PPMO) infusion (e.g., about 0.5-100 mg/kg/week). Alternatively, subjects suspected of having or diagnosed as having DMD or BMD receive weekly intravenous administration of H-D-Arg-2′6′-Dmt-Lys-Phe-NH₂ (SEQ ID NO: 2) or H-D-Arg-2′6′-Dmt-Lys-Phe-OH (SEQ ID NO: 3) (e.g. with 0.05-1.0 mg/kg/hr. for up to 4 hours) and weekly intravenous (IV) PMO (or PPMO) infusion (e.g., about 0.5-100 mg/kg/week). In some instances, the subjects may already be receiving PMO or PPMO therapy. Subjects will be regularly evaluated (e.g., weekly, bi-weekly, monthly, etc.) for the presence and/or severity of signs and symptoms of muscular dystrophy associated with DMD or BMD including, but not limited to, dystrophin expression levels in skeletal, cardiac, and/or smooth muscle. Treatments will be maintained at least until such a time as one or more signs or symptoms of DMD or BMD are ameliorated or eliminated. The study may be conducted in a randomized withdrawal trial (e.g., randomized, double-blind, placebo-controlled withdrawal trial) to assess the impact of the peptide in combination with a PMO or PPMO on the subjects followed by the impact of their withdrawal relative to a control group still receiving the peptide and PMO or PPMO.

### Results

It is predicted that subjects suspected of having or diagnosed as having DMD or BMD and receiving therapeutically effective amounts of H-D-Arg-2′6′-Dmt-Lys-Phe-NH₂ (SEQ ID NO: 2) or H-D-Arg-2′6′-Dmt-Lys-Phe-OH (SEQ ID NO: 3) in combination with PMO or PPMO therapy will display reduced severity or elimination of one or more signs or symptoms of DMD or BMD. These results are anticipated to show that H-D-Arg-2′6′-Dmt-Lys-Phe-NH₂ (SEQ ID NO: 2) or H-D-Arg-2′6′-Dmt-Lys-Phe-OH (SEQ ID NO: 3) in combination with PMO or PPMO therapy is useful in increasing dystrophin expression levels in the subjects as compared to untreated controls or control subjects receiving PMO or PPMO therapy alone (i.e., not receiving treatment with H-D-Arg-2′6′-Dmt-Lys-Phe-NH₂ (SEQ ID NO: 2) or H-D-Arg-2′6′-Dmt-Lys-Phe-OH (SEQ ID NO: 3)). In some cases, it is predicted that expression of dystrophin will be significantly improved in the cardiac muscles. In some cases, it is predicted that the improvements in dystrophin expression within the subject will lead to increased life expectancy in subjects suffering from muscular dystrophy.

Accordingly, these results will demonstrate that H-D-Arg-2′6′-Dmt-Lys-Phe-NH₂ (SEQ ID NO: 2) or H-D-Arg-2′6′-Dmt-Lys-Phe-OH (SEQ ID NO: 3) in combination with PMO or PPMO therapy is useful in methods for treating or delaying the onset of muscular dystrophy in subjects suspected of having or diagnosed as having DMD or BMD.

Other signs or symptoms of DMD or BMD that might be evaluated and result in improvements include:
Improvements in the subject's ambulation (e.g. delay in onset or progression of the subject's decline in ambulation or outright improvement in the subject's ability to move)
Delayed motor development
Enlarged calf muscles (pseudohypertrophy)
Muscle weakness that gets worse over time
Toe walking or waddling gait
Using hands to get up off the floor (Gower's maneuver)
Progressive enlargement of the heart (cardiomyopathy)
Delay in the progression of left ventricle dilation
Delay in the progression of left ventricle fibrosis
Delay in the reduction of left ventricle stroke volume
Improvements in left ventricle end diastolic volume (LVEDV)
Improvements in left ventricle end systolic volume (LVESV)
Change in myocardial strain
Improvements in respiratory function
Abnormal urinary color
Elevated serum creatine kinase
Exercise intolerance

### Example 3—One-Step Synthesis of 1-((R)-4-ammonio-5-(((S)-1-(((S)-6-ammonio-1-(((S)-1-carboxy-2-phenylethyl)amino)-1-oxohexan-2-yl)amino)-3-(4-hydroxy-2,6-dimethylphenyl)-1-oxopropan-2-yl)amino)-5-oxopentyl)guanidinium chloride (A-2, tris-HCl salt) from elamipretide (A-1, tris acetate salt)

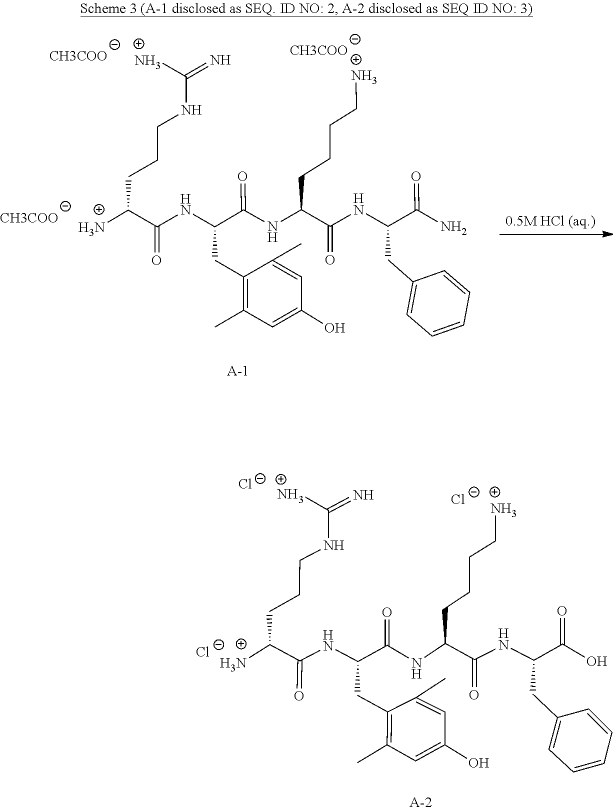

A solution of SBT-031 triacetate (8.2 g, 10 mmol) in 0.5M aq. hydrochloric acid was stirred at 35-40° C. for 5 days. Then solvent was removed under reduced pressure and crude product purified by reversed phase flash chromatography (water (pH=3)/MeCN, from 0.25 to 4%) to give A-2 (5.6 g) in 75% yield. [0248]¹H-NMR (400 MHz, Methanol-d4) δ 7.37-7.14 (m, 5H), 6.43 (s, 2H), 4.80 (dd, J=9.5, 6.9 Hz, 1H), 4.64 (dd, J=8.6, 5.2 Hz, 1H), 4.37 (dd, J=8.1, 5.9 Hz, 1H), 4.01 (t, J=6.3 Hz, 1H), 3.21 (dd, J=14.0, 5.2 Hz, 1H), 3.16-2.88 (m, 7H), 2.27 (s, 6H), 1.85-1.54 (m, 6H), 1.53-1.20 (m, 4H).

### EQUIVALENTS

The present technology is not to be limited in terms of the particular embodiments described herein, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Various embodiments are set forth within the following claims.

1. A method for treating muscular dystrophy, comprising administering to the subject a therapeutically effective amount of a phosphorodiamidate morpholino oligomer (PMO) or a peptide-conjugated PMO (PPMO) in combination with a peptide of formula A: 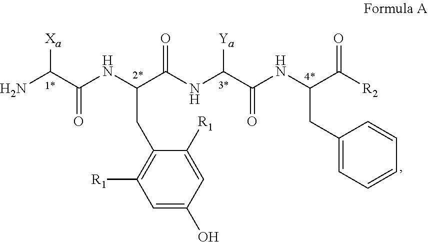
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 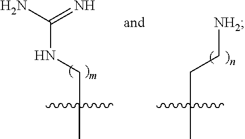
each m is 2, 3 or 4; each n is independently 1, 2, or 3; and the absolute stereochemistry at each of stereocenters 1*, 2*, 3*, and 4* is independently D or L.

2. The method of claim 1, wherein the peptide of generic Formula A is a peptide of formula A-1: 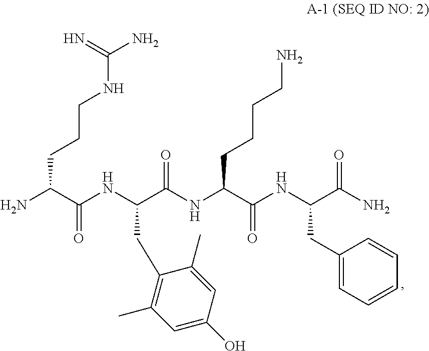 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]
or a pharmaceutically acceptable salt, hydrate, solvate and/or tautomer thereof.

3. The method of claim 1, wherein the peptide of generic Formula A is a peptide of formula A-2: 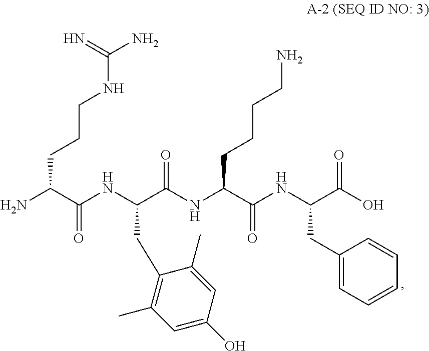 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]
or a pharmaceutically acceptable salt, hydrate, solvate and/or tautomer thereof.

4. The method of claim 1, wherein the peptide of generic Formula A is a peptide of formula A-3, A-4, A-5, A-6, A-7 or A-8: 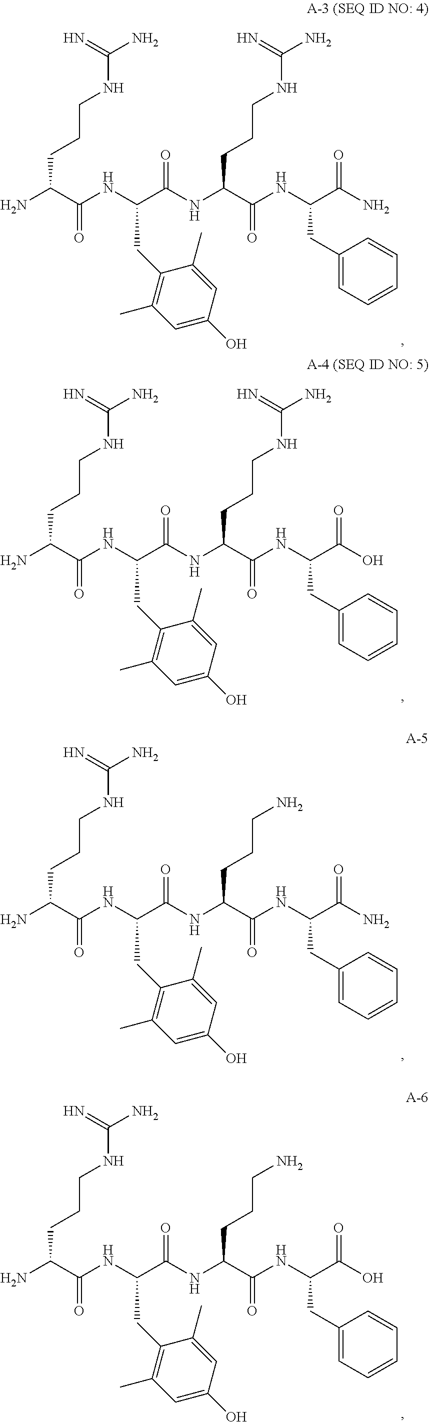 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O ; Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O] 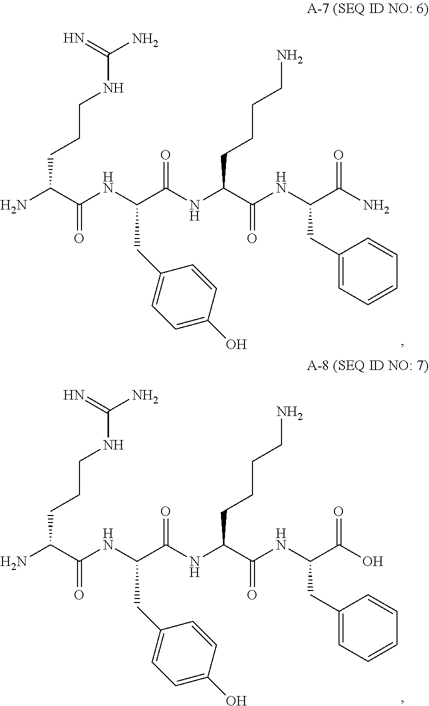 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O ; N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]
or a pharmaceutically acceptable salt, hydrate, solvate, and/or tautomer thereof.

5. The method of claim 1, wherein treatment reduces, ameliorates, and/or delays the onset of muscular dystrophy.

6. The method of claim 1, wherein the administration increases the level of dystrophin expression in the subject compared to a control subject.

7. The method of claim 6, wherein the administration increases dystrophin expression in skeletal, cardiac, and/or smooth muscle in the subject as compared to an untreated subject (or untreated control group of subjects) or as compared to a subject (or control group of subjects) administered the PMOs and/or PPMOs alone or the peptide (or mixture of peptides) alone.

8. The method of claim 1, wherein the muscular dystrophy is Duchenne muscular dystrophy.

9. The method of claim 1, wherein the muscular dystrophy is Becker's muscular dystrophy.

10. The method of claim 1, wherein the peptide is administered daily for: (i) 24 weeks or more; (ii) 48 weeks or more; (iii) 72 weeks or more; or (iv) 96 weeks or more.

11. The method of claim 1, wherein the PMO or PPMO is administered once weekly for: (i) 24 weeks or more; (ii) 48 weeks or more; (iii) 72 weeks or more; or (iv) 96 weeks or more.

12. The method of claim 1, wherein the peptide and PMO or PPMO are administered orally, topically, systemically, intraperitoneally, subcutaneously, intravenously, intradermally, transdermally, ophthalmically, intrathecally, intracerebroventricularly, iontophoretically, transmucosally, intravitreally, intranasally, or intramuscularly.

13. The method of claim 12, wherein the PMO or PPMO is administered intravenously.

14. The method of claim 12, wherein the peptide is administered subcutaneously.

15. The method of claim 1, wherein the subject is human.

16. The method of claim 1, further comprising separately, sequentially, or simultaneously administering an additional therapeutic agent to the subject.

17. The method of claim 1, wherein the PMO is selected from the group consisting of Eteplirsen (Exondys 51®), Golodirsen (Vyondys 53™), Viltolarsen (Viltepso®), and Casimersen (Amondys 45™).

18. The method of claim 1, wherein the peptide and the PMO or PPMO are administered intravenously.

19. The method of claim 18, wherein the peptide and the PMO or PPMO are administered simultaneously.

20. The method of claim 1, wherein the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

21. The method of claim 1, wherein the peptide of Formula A is administered in a depot formulation.

22. The method of claim 21, wherein the depot formulation comprises the peptide of Formula A encapsulated or otherwise disposed in silica microparticles.

23. The method of claim 21, wherein the depot formulation is a sustained release depot formulation.

24. The method of claim 23, wherein the peptide of Formula A is released in an effective amount over days, weeks or months.

25. A composition comprising:
a) a peptide of formula A: 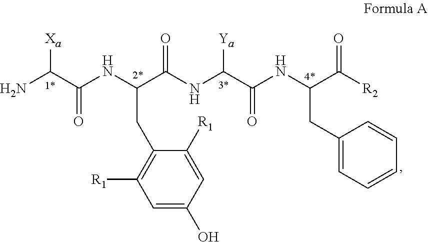
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 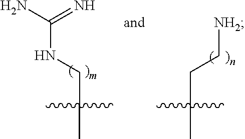
each m is 2, 3 or 4; each n is independently 1, 2, or 3; and the absolute stereochemistry at each of stereocenters 1*, 2*, 3*, and 4* is independently D or L; and
b) a phosphorodiamidate morpholino oligomer (PMO) or a peptide-conjugated PMO (PPMO).

26.-32. (canceled)

33. A method for augmenting the production of dystrophin in a mammalian subject in need thereof, comprising administering to the subject a therapeutically effective amount of a phosphorodiamidate morpholino oligomer (PMO) or a peptide-conjugated PMO (PPMO) in combination with a peptide of formula A: 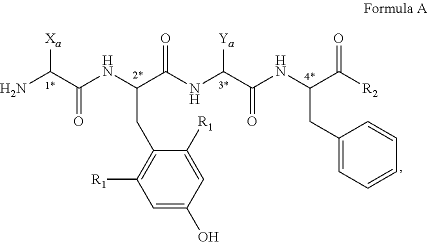
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 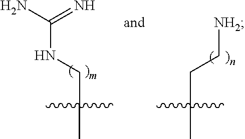
each m is 2, 3 or 4; each n is independently 1, 2, or 3; and the absolute stereochemistry at each of stereocenters 1*, 2*, 3*, and 4* is independently D or L,
wherein the administration increases dystrophin expression in skeletal, cardiac, and or smooth muscle in the subject as compared to an untreated subject (or untreated control group of subjects) or as compared to a subject (or control group of subjects) administered the PMO and/or PPMO alone or the peptide alone.

34.-55. (canceled)


SEQUENCE LISTING (7 sequences)

SEQ ID NO: 1 - DNA, 25 bp, synthetic construct
  source 1..25: mol_type = other DNA
  modified_base 1..25: mod_base = OTHER; note = Phosphorodiamidate morpholino backbone
ggccaaacctcggcttacctgaaat

SEQ ID NO: 2 - protein, 4 aa, synthetic construct
  SITE 1: note = D-amino acid
  SITE 2: note = 2,6-Dimethyl-L-tyrosine
  SITE 4: note = Amidated residue
RYKF

SEQ ID NO: 3 - protein, 4 aa, synthetic construct
  SITE 1: note = D-amino acid
  SITE 2: note = 2,6-Dimethyl-L-tyrosine
RYKF

SEQ ID NO: 4 - protein, 4 aa, synthetic construct
  SITE 1: note = D-amino acid
  SITE 2: note = 2,6-Dimethyl-L-tyrosine
  SITE 4: note = Amidated residue
RYRF

SEQ ID NO: 5 - protein, 4 aa, synthetic construct
  SITE 1: note = D-amino acid
  SITE 2: note = 2,6-Dimethyl-L-tyrosine
RYRF

SEQ ID NO: 6 - protein, 4 aa, synthetic construct
  SITE 1: note = D-amino acid
  SITE 4: note = Amidated residue
RYKF

SEQ ID NO: 7 - protein, 4 aa, synthetic construct
  SITE 1: note = D-amino acid
RYKF